(19) United States
(12) Patent Application Publication
(10) Pub. No.: US 2014/0087970 A1
(43) Pub. Date: Mar. 27, 2014

(54) SERINE BIOSYNTHESIS PATHWAY INHIBITION FOR TREATMENT OF CANCER

(71) Applicants: Richard Possemato, Brighton, MA (US); David M. Sabatini, Cambridge, MA (US)

(72) Inventors: Richard Possemato, Brighton, MA (US); David M. Sabatini, Cambridge, MA (US)

(73) Assignee: Whitehead Institute for Biomedical Research, Cambridge, MA (US)

(21) Appl. No.: 14/008,873

(22) PCT Filed: Mar. 30, 2012
(86) PCT No.: PCT/US2012/031599

§ 371 Date: Dec. 11, 2013

Related U.S. Application Data
(60) Provisional application No. 61/469,577, filed on Mar. 30, 2011.

(51) Int. Cl. C12N 15/10; G01N 33/573; C12Q 1/68

(52) U.S. Cl. CPC C12N 15/1082; C12Q 1/6881; G01N 33/573
USPC 506/10; 435/6.13; 435/7.4; 506/14

(57) ABSTRACT

In some aspects, the invention provides compounds and methods of use for treating tumors. In some aspects, the methods comprise administering a serine biosynthesis pathway inhibitor to a subject, wherein the subject has a tumor that overexpresses PHGDH In some embodiments, the tumor is an ER negative breast cancer. In some aspects, the invention provides an in vivo RNAi-based negative selection screen of use to identify drug targets for treatment of tumors.

### RELATED APPLICATIONS

This application claims the benefit of, and priority to, U.S. Provisional Application Ser. No. 61/469,577, filed Mar. 30, 2011, the entire teachings of which are incorporated herein by reference.

### GOVERNMENT SUPPORT

This invention was made with government support under CA 103866 awarded by the National Institutes of Health. The government has certain rights in the invention.

### BACKGROUND OF THE INVENTION

Cancer is a leading cause of death worldwide and accounted for approximately 7.6 million deaths (around 13% of all deaths) in 2008 according to the World Health Organization. There is a significant need for new therapeutic approaches for the treatment of cancer. There is also a need for new methods for identifying promising drug targets for treatment of cancer. There is also a need for methods of identifying patients who are likely to benefit from particular treatment approaches.

### SUMMARY OF THE INVENTION

The invention provides, among other things, RNAi-based negative selection screening methods of use, e.g., to identify drug targets for anticancer drug development In some aspect, the invention provides drug targets identified using the inventive screening methods.

In some aspects, the invention provides methods of treating cancer comprising administering a serine biosynthesis pathway (SBP) inhibitor to a subject in need thereof. In some embodiments, the SBP inhibitor is a PHGDH inhibitor.

The invention further provides an immunohistochemical assay that can be used to identify tumors that overexpress PHGDH. In some aspects, such an assay is of use to identify tumors that are responsive to treatment with an SBP inhibitor, e.g., a PHGDH inhibitor.

The practice of the present invention will typically employ, unless otherwise indicated, conventional techniques of molecular biology, cell culture, recombinant nucleic acid (e.g., DNA) technology, immunology, nucleic acid and polypeptide synthesis, detection, manipulation, and quantification, and RNA interference that are within the skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Further information on cancer may be found in Cancer: Principles and Practice of Oncology (V. T. De Vita et al., eds., J.B. Lippincott Company, 7th ed., 2004 or 8th ed., 2008) and Weinberg, R A, The Biology of Cancer, Garland Science, 2006. All patents, patent applications, publications, references, databases, websites, etc., cited in the instant patent application are incorporated by reference in their entirety. In the event of a conflict or inconsistency with the specification, the specification shall control. The Applicants reserve the right to amend the specification based on any of the incorporated references and/or to correct obvious errors. None of the content of the incorporated references shall limit the invention.

### BRIEF DESCRIPTION OF THE DRAWINGS

FIGS. 1A-1E. Outline of in vivo pooled screening strategy identifying PHGDH as essential for tumorigenesis. a, Venn Diagram showing the number of genes transcriptionally upregulated in advanced breast cancer (red circle), broadly upregulated across multiple cancer types versus normal tissues (green circle) or associated with stem cell markers (blue circle). The number of genes that overlap between the three groups are indicated at the intersection of the circles. b, Screening outline. Short hairpin RNAs (shRNAs) were expressed in pools in MCF10DCIS.com cells and injected orthtotopically into immune compromised mice. Genomic DNA (gDNA) harvested before or after growth in culture or in vivo was subjected to massively parallel DNA sequencing to determine the changes in shRNA abundance. c, Log(2) fold change in the abundance of each shRNA tested (blue diamonds) or neutral shRNAs (red squares) is shown for a single tumor (X-axis) compared to an average of eleven tumors (Y-axis), d, Table of genes that scored in the in vivo screen. e, The average weight of tumors injected into immune compromised mice are reported for MCF10DCIS.com cells expressing shRNAs targeting PHGDH (PHGDH_{—}1, PHGDH_{—}2 and PHGDH_{—}3) or a control shRNA (GFP) and protein expression of PHGDH or RPS6 (S6) in these cells grown in vitro. The average+/−SEM are reported for four tumors from each class. The asterisk indicates a probability value (p) of less than 0.05. ND=Not Done.

FIGS. 2A-2G. Genomic amplifications of PHGDH in cancer and association of PHGDH expression with aggressive breast cancer markers. a, Copy number data for 111 melanoma samples including 108 cell lines or short term cultures (left box) and 243 breast cancer samples including 50 cell lines (right box) over the 2 MB region of chromosome 1 specified by the ideogram of chromosome 1 at the right. The colored bar at the top indicates the degree of copy number loss (blue) or gain (red). Samples are organized in columns and sorted by copy number at the PHGDH locus (horizontal dotted lines). The identity and location of other genes in the region depicted are shown on the left. To the left of each box is a graph showing the significance of the amplification over this region (on a scale of −log₁₀(q-value)). A value of 0.25 (or ˜0.60 on the −log₁₀ scale) is considered the threshold to identify a significantly amplified region. b, Gene expression data is shown from one study investigating the expression of PHGDH in normal breast tissue or in all breast cancer, or in breast cancer classified by estrogen receptor (ER) status. c, Representative gene expression data is shown from four studies investigating the association of PHGDH gene expression with common breast cancer markers or classifications including molecular subtypes (Luminal versus Basal), histopathological subtypes (Grade), estrogen receptor status (ER-positive versus ER-negative) and five-year survival (living versus deceased). Whiskers indicate the 91^{st} and 9^{th} percentile for each group and outliers are not shown. d, The table reports the number of human breast cancer samples with “weak”, “moderate”, or “strong” staining for PHGDH from four breast cancer subgroups as defined by their estrogen receptor (ER) or human epidermal growth factor receptor 2 (Her2) status. The images at the right demonstrate representative human breast cancer specimens of the three staining intensities. The asterisk indicates a p-value of (P<0.0001) for the comparison of staining intensities for ER-positive versus ER-negative samples (Fisher's exact test). e, PHGDH protein levels are shown for four cell lines with and five cell lines without PHGDH genomic amplification (annotated with a “+” or “−”). Immunoflourescent quantification (LI-COR) of PHGDH levels relative to RPS6 (S6) and normalized to MCF-10A and MCF7 are shown as numbers below the PHGDH immunoblot. f, PHGDH protein levels are shown for MCF7 cells or two ER-negative cell lines with elevated PHGDH expression, but without PHGDH amplification in the same format as (e). g, PHGDH protein levels for MCF-10A derived cells are depicted in the same format as (e).

FIGS. 3A-3H. Cell lines with elevated PHGDH expression are sensitive to PHGDH suppression. a, Schematic diagram of serine biosynthesis pathway b-d (not included herein) e, Immunoblots of PHGDH and RPS6 (S6) are shown at left for the indicated cell lines expressing a control shRNA (GFP) or shRNAs against PHGDH (PHGDH_{—}1 and PHGDH_{—}2). Bars indicate the relative proliferation rate of cells transduced with these shRNA constructs after seven days of growth. f, Images showing the cellular morphology of MDA-MB-468 cells seven days after transduction with a control shRNA (shGFP) or an shRNA targeting PHGDH (shPHGDH_{—}1 and shPHGDH_{—}2). Cells in the lower two panels are largely detached from the plate. g, Immunoblots of PSPH, PSAT1 and RPS6 (S6) are shown at right for the indicated cell lines expressing a control shRNA (GFP) or shRNAs against PSAT1 (PSAT1_{—}1 and PSAT1_{—}2) or PSPH (PSPH_{—}1 and PSPH_{—}2). Bars indicate the relative proliferation rate of cells transduced with these shRNA constructs after seven days of growth. h, In vivo tumor growth of MDA-MB-468 cells expressing a doxycycline inducible control shRNA (GFP) or shRNA against PHGDH (shPHGDH_{—}2) in mice fed a doxycycline (Dox, 2 mg/kg, green lines, n=5) or normal (blue lines, n=4) diet. Tumors were allowed to form until palpable before introduction of doxycycline diet. Immunoblots of PHGDH or RPS6 (S6) are shown for cells grown in vitro. For all graphs the asterisk indicates a probability value (p)<0.05 relative to the control. Error bars for tumor size indicate standard error and for cell number measurements indicate standard deviation (n=3).

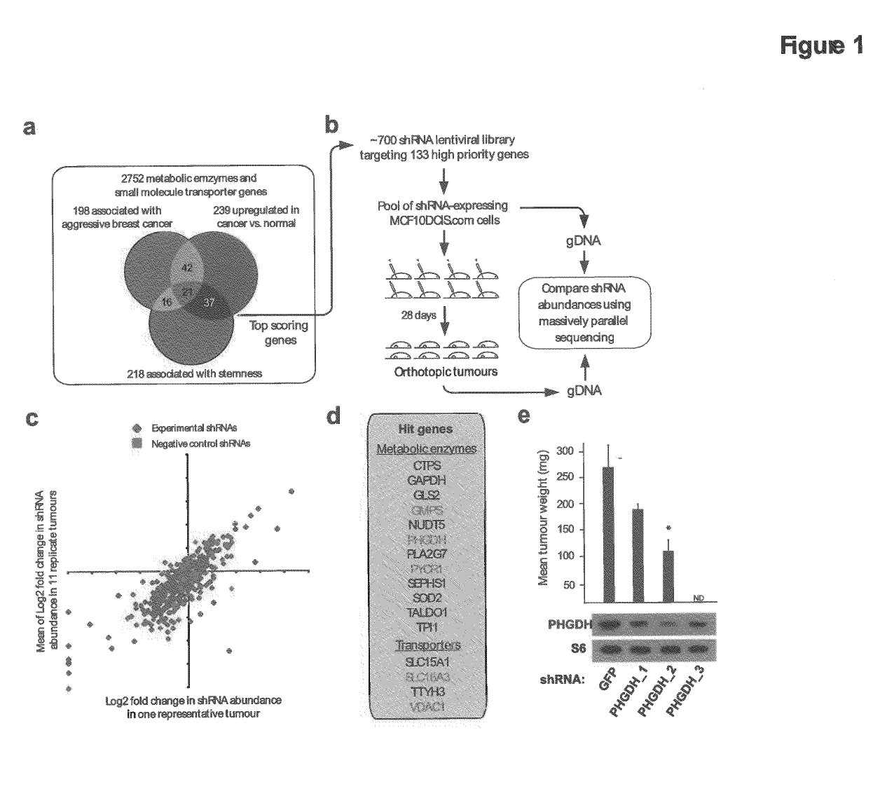

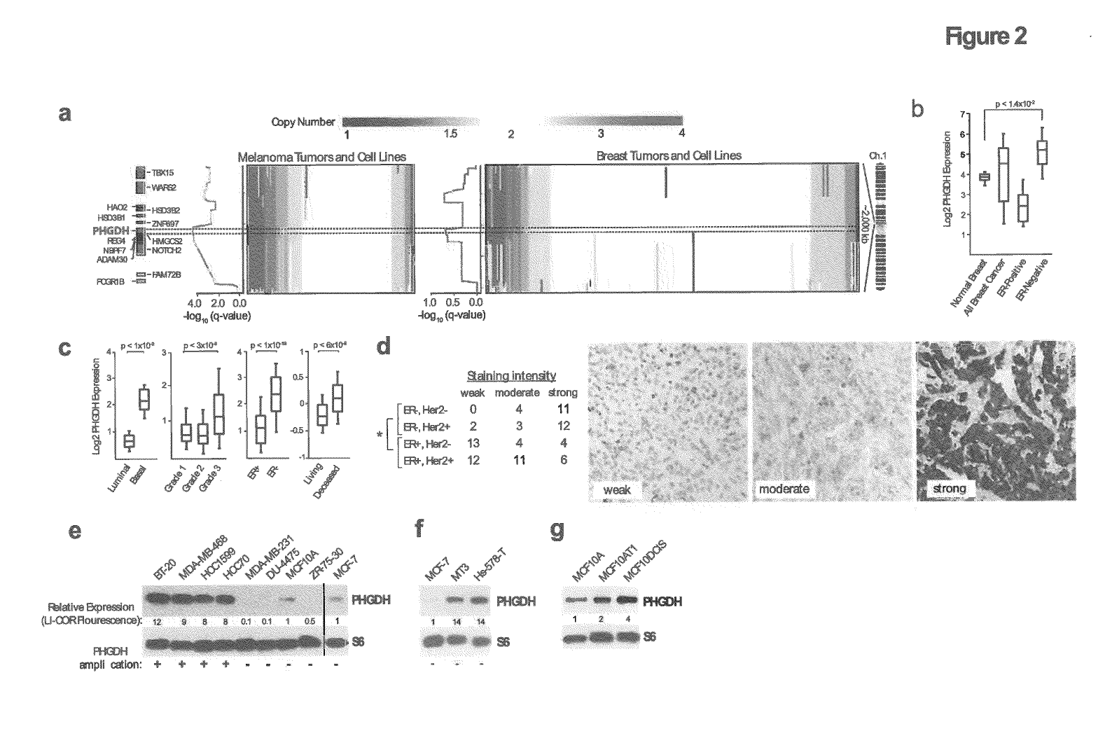

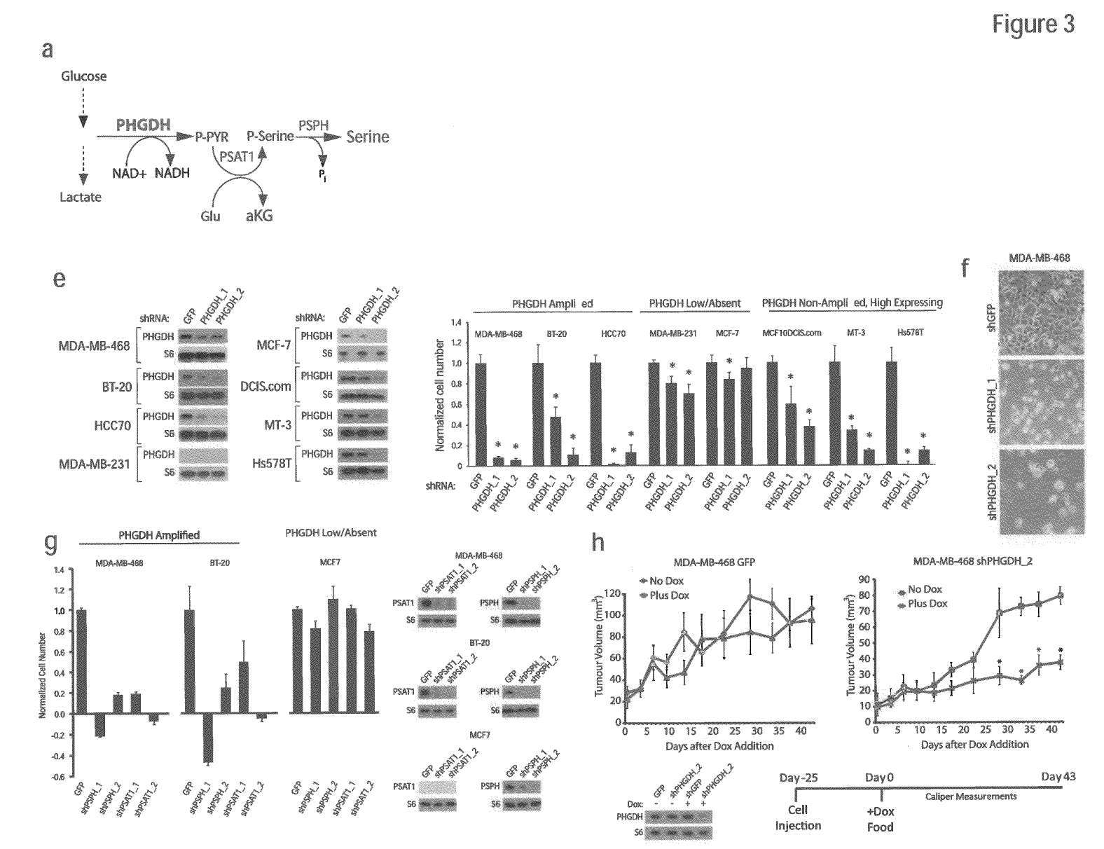

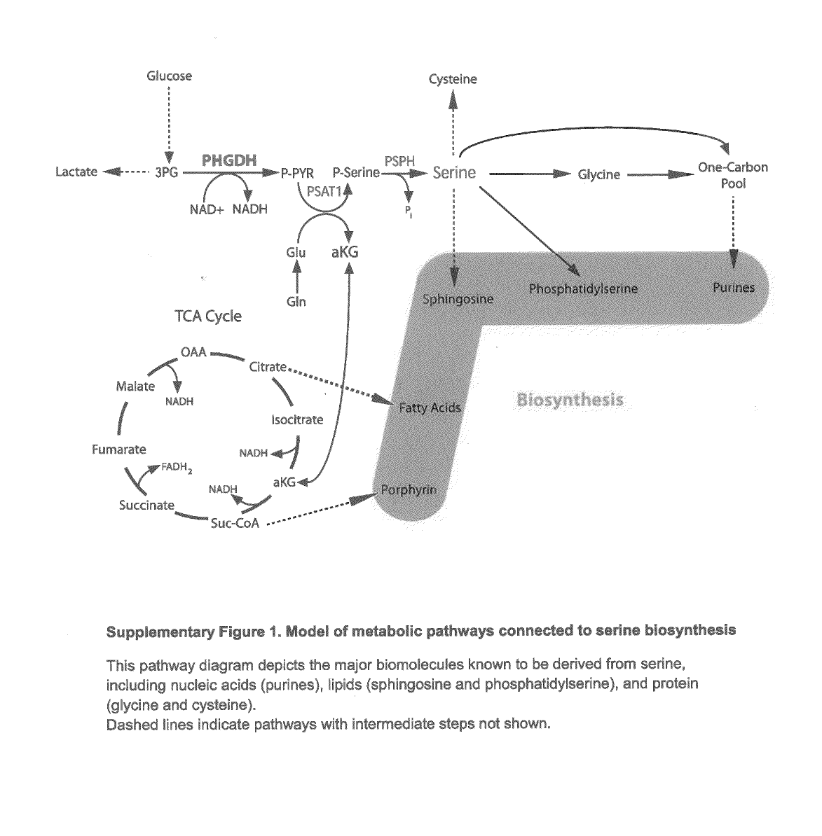

Supplementary FIG. 1. Schematic diagram of the serine biosynthesis pathway and model of metabolic pathways connected to serine biosynthesis This pathway diagram depicts the major biomolecules known to be derived from serine, including nucleic acids (purines), lipids (sphingosine and phosphatidylserine), and amino acids (glycine and cysteine). Dashed lines indicate pathways with intermediate steps not shown.

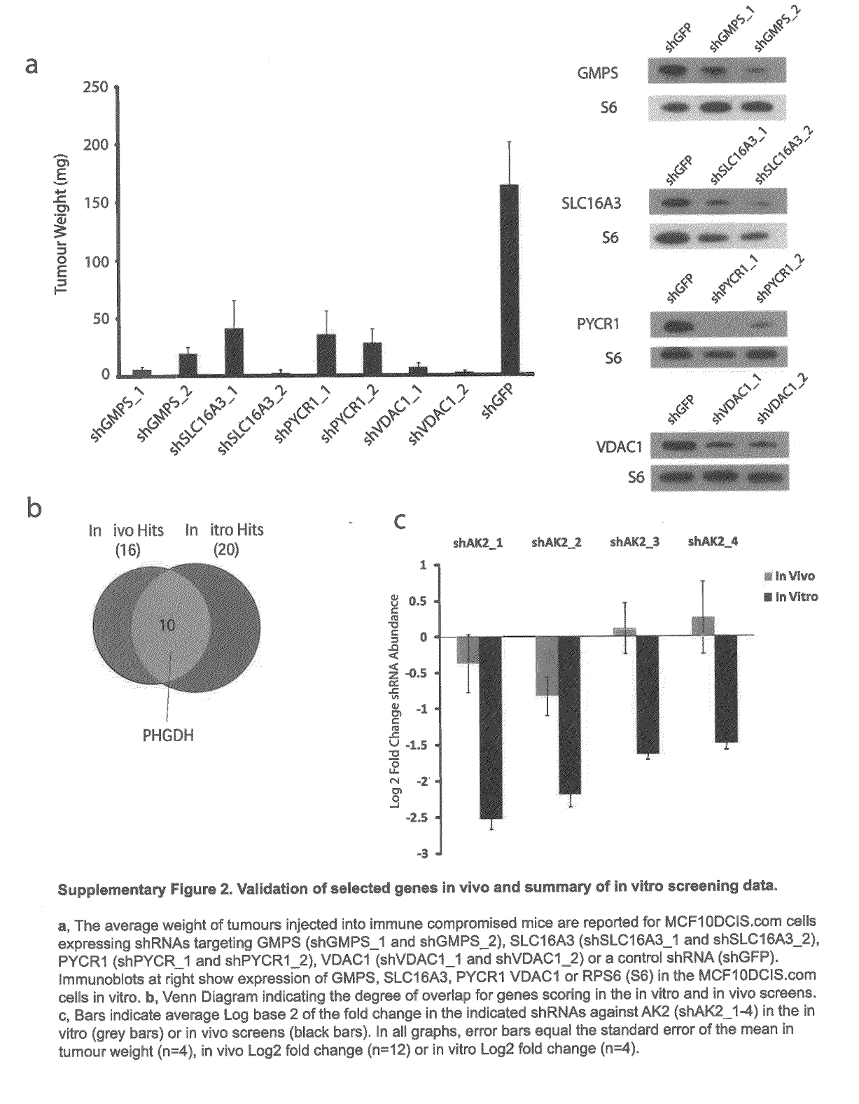

Supplementary FIG. 2 (A-C). Validation of selected genes in vivo and summary of in vitro screening data. a, The average weight of tumors injected into immune compromised mice are reported for MCF 10DCIS.com cells expressing shRNAs targeting GMPS (shGMPS_{—}1 and shGMPS_{—}2), SLC16A3 (shSLC16A3_{—}1 and shSLC16A3_{—}2), PYCR1 (shPYCR_{—}1 and shPYCR1_{—}2), VDAC1 (shVDAC1_{—}1 and shVDAC1_{—}2) or a control shRNA (shGFP). Immunoblots at right show expression of GMPS, SLC16A3, PYCR1 VDAC1 or RPS6 (S6) in the MCF10DCIS.com cells in vitro. b, Venn Diagram indicating the degree of overlap for genes scoring in the in vitro and in vivo screens. c, Bars indicate average Log base 2 of the fold change in the indicated shRNAs against AK2 (shAK2_{—}1-4) in the in vitro (grey bars) or in vivo screens (black bars). In all graphs, error bars equal the standard error of the mean in tumor weight (n=4), in vivo Log 2 fold change (n=12) or in vitro Log 2 fold change (n=4).

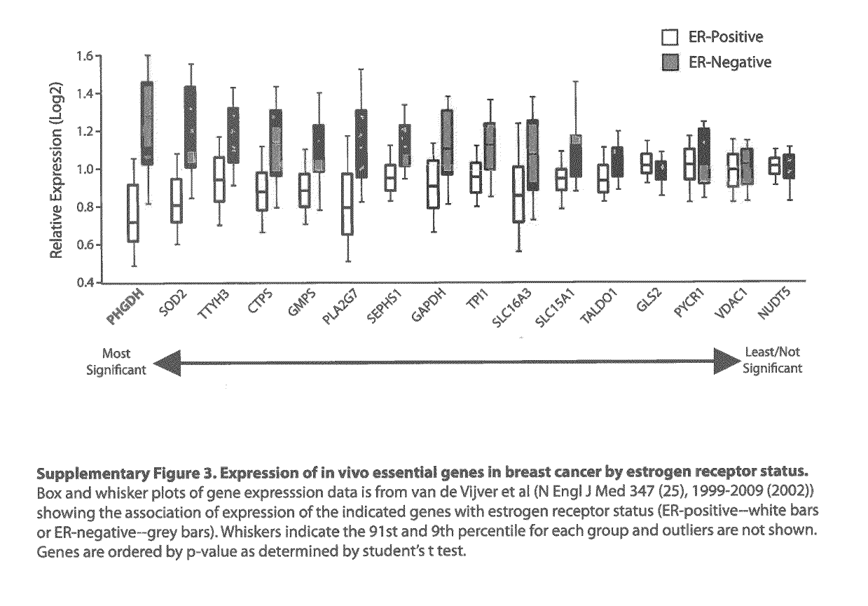

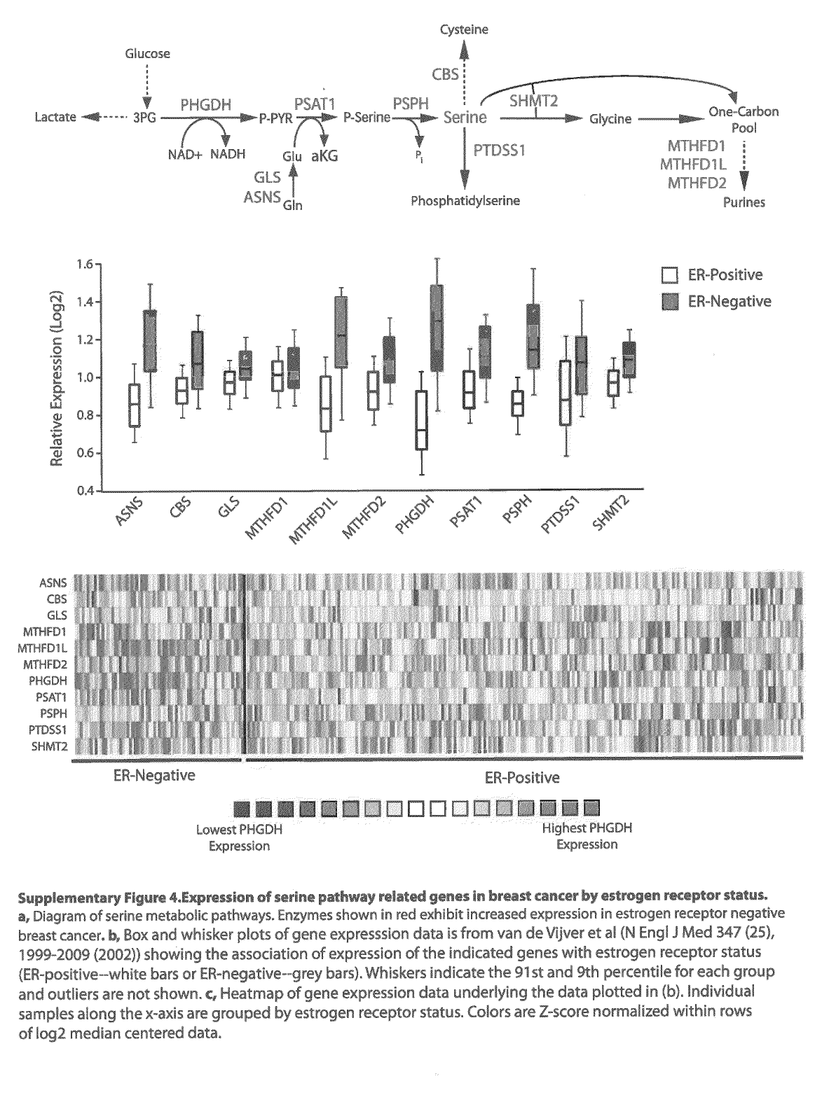

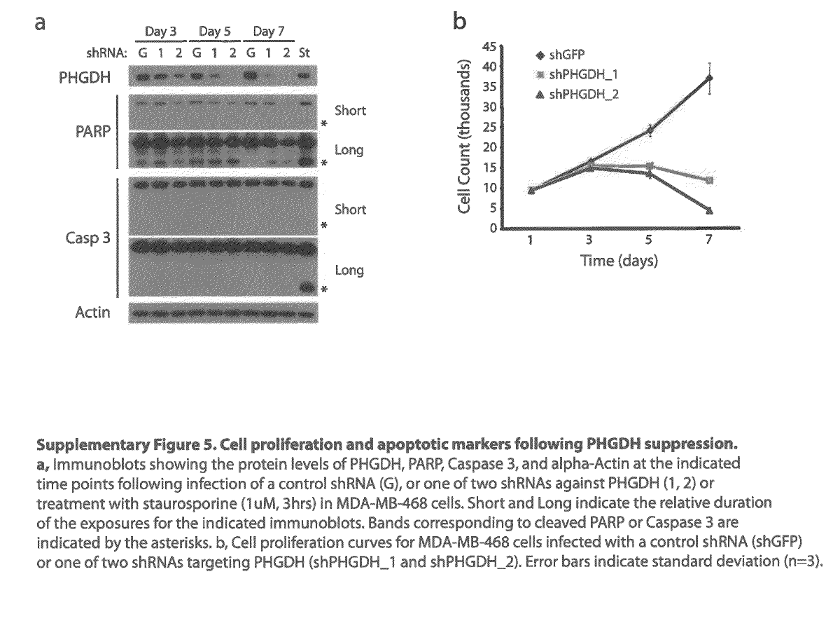

Supplementary FIG. 3. Expression of in vivo essential genes in breast cancer by estrogen receptor status. Box and whisker plots of gene expresssion data is from van de Vijver et at (N Engl J Med 347 (25), 1999-2009 (2002)) showing the association of expression of the indicated genes with estrogen receptor status (ER-positive—white bars or ER-negative—grey bars). Whiskers indicate the 91st and 9th percentile for each group and outliers are not shown. Genes are ordered by p-value as determined by student's t test.

Supplementary FIG. 4. Expression of serine pathway related genes in breast cancer by estrogen receptor status. a, Diagram of serine metabolic pathways. Enzymes shown in red exhibit increased expression in estrogen receptor negative breast cancer. b, Box and whisker plots of gene expresssion data is from van de Vijver et al (N Engl J Med 347 (25), 1999-2009 (2002)) showing the association of expression of the indicated genes with estrogen receptor status (ER-positive—white bars or ER-negative—grey bars). Whiskers indicate the 91st and 9th percentile for each group and outliers are not shown. c, Heatmap of gene expression data underlying the data plotted in (b). Individual samples along the x-axis are grouped by estrogen receptor status, Colors are Z-score normalized within rows of log 2 median centered data.

Supplementary FIG. 5. Cell proliferation and apoptotic markers following PHGDH suppression. a, Immunoblots showing the protein levels of PHGDH, PARP, Caspase 3, and alpha-Actin at the indicated time points following infection of a control shRNA (G), or one of two shRNAs against PHGDH (1, 2) or treatment with staurosporine (1 uM, 3 hrs) in MDA-MB-468 cells. Short and Long indicate the relative duration of the exposures for the indicated immunoblots. Bands corresponding to cleaved PARP or Caspase 3 are indicated by the asterisks. b, Cell proliferation curves for MDA-MB-468 cells infected with a control shRNA (shGFP) or one of two shRNAs targeting PHGDH (shPHGDH1_{—}1 and shPHGDH_{—}2). Error bars indicate standard deviation (n=3).

### DETAILED DESCRIPTION OF CERTAIN EMBODIMENTS OF THE INVENTION

I. In Vivo RNAi-Based Screening Methods

In some aspects, the invention provides in vivo RNAi-based screening methods. In some embodiments, the in vivo RNAi-based screening methods entail a negative selection approach that allows for the identification of genes involved in and/or essential for tumorigenesis. In some embodiments, the inventive screening methods comprise identifying RNAi agents (e.g., short hairpin RNAs) whose expression impairs the ability of tumorigenic cells to survive and/or proliferate in vivo. Tumorigenic cells harboring different RNAi agents (inhibiting expression of different genes) are introduced into an animal host. The animal host is maintained for a period of time sufficient for such cells to give rise to a tumor. The tumor (or a sample thereof) is isolated, RNAi agents within the isolated tumor or sample thereof are identified and the abundance of each of multiple RNAi agents is compared with the abundance of that RNAi agent in the cells that were introduced into the animal host. RNAi agents whose expression inhibits survival and/or proliferation of tumors cells harboring them are underrepresented in the tumor (since the cells containing such RNAi agents will be at a selective disadvantage). The genes whose expression is inhibited by such RNAi agents, and the products (e.g., proteins) encoded by these genes, represent potential targets for development of pharmacological agents for treatment of cancer and are sometimes referred to herein as “drug targets”. “Drug target” is used herein consistently with usage in the art, and encompasses molecules (e.g., a biomolecule produced by a cell, such as a protein) that is involved directly or indirectly in a disease process and/or whose modulation (e.g., inhibition or activation of its expression or activity, whether by direct or indirect means) is of use or reasonably likely to be of use to treat a disease. In some aspects, a drug target is a molecule involved in a metabolic or signaling pathway that is at least somewhat specific to a disease (e.g., at least somewhat specific to a subject in diseased state or at least somewhat specific to diseased tissue) or functions abnormally (or fails to function normally) in at least some cells or tissues of an individual having the disease, as compared with individuals not having the disease. In some embodiments, a drug target is a molecule whose inhibition is of use or reasonably likely to be of use to treat a disease. For example, inhibitors of various drug targets described herein are candidate agents for treatment of tumors. For purposes of description herein, the term “drug target” may be used to refer to a biomolecule (e.g., a protein) with which a modulating agent such as an inhibitor physically interacts and/or to a gene that encodes the biomolecule. The term “tumor” is used herein interchangeably with “cancer”. In many embodiments, a tumor is a malignant tumor. In some embodiments, a tumor is a carcinoma. In some embodiments, a tumor is a sarcoma.

The term “RNAi agent” encompasses nucleic acids that can be used to achieve RNA interference (RNAi) in mammalian cells. RNAi, as known in the art, encompasses processes in which sequence-specific silencing of gene expression is effected by an RNA-induced silencing complex (RISC) that has a short RNA strand incorporated therein, which strand directs or “guides” sequence-specific degradation or translational repression of mRNA to which it has complementarity. The complementarity between the short RNA and mRNA need not be perfect (100%) but need only be sufficient to result in inhibition of gene expression. For example, the degree of complementarity and/or the characteristics of the structure formed by hybridization of the mRNA and the short RNA strand can be such that the strand can (i) guide cleavage of the mRNA in the RNA-induced silencing complex (RISC) and/or (ii) cause translational repression of the mRNA by RISC. RNAi may be achieved artificially in eukaryotic, e.g., mammalian, cells in a variety of ways. For example, RNAi may be achieved by introducing an appropriate short double-stranded nucleic acid into the cells or expressing in the cells a nucleic acid that is processed intracellularly to yield such short dsRNA. Exemplary RNAi agents include short hairpin RNA (shRNA), a short interfering RNA (siRNA), microRNA (miRNA) and a miRNA precursor.

In some aspects, the invention provides a method of identifying a potential drug target for antitumor therapy, the method comprising: (a) providing a pool of cells comprising multiple distinct populations of tumorigenic cells, wherein each of at least 5 distinct populations harbors in its genomic DNA an expression cassette encoding an RNAi agent that has sequence correspondence to a different gene; (b) introducing the pool of cells into an animal host; (c) maintaining the animal host for a sufficient time period for a tumor to develop under conditions in which the RNAi agents are expressed during at least part of the time period; (d) harvesting at least a portion of the tumor; and (e) identifying an RNAi agent that became significantly depleted during tumor formation as compared with its abundance in the pool of cells of step (b), wherein the gene to which such RNAi agent has sequence correspondence is identified as a potential drug target for antitumor therapy. In some embodiments of an inventive screening method, the RNAi agents comprise shRNAs.

In some embodiments, expression of the RNAi agents is regulatable, e.g., inducible. Any of a variety of regulatable expression control elements (e.g., inducible or repressible promoters) known in the art can be used. In some embodiments, expression is regulatable (e.g, inducible) using a small molecule. For example, tetracycline or a tetracycline analog such as doxycycline can be used. In other embodiments, a hormone or metal is used as an inducing agent. Cells may be maintained in culture in the absence of expression of the RNAi agent. Expression of the RNAi agent may be induced following introduction of the cells into an animal host.

In some embodiments, cells harboring RNAi agents in their genome are generated by infection with virus vectors, e.g., retrovirus vectors (e.g., lentiviruses) harboring the expression cassette in their genomic nucleic acid. In other embodiments, stable cell lines harboring RNAi agents in their genomes may be produced by transfection with a vector such as a plasmid (which may in some embodiments comprise at least a portion of a viral genome).

Any of a wide variety of tumorigenic cells can be used in the inventive screening methods. For example, tumorigenic cells can be derived from a brain, bladder, breast, cervical, colon, endothelial, epithelial, lung, mesothelial, ovarian, pancreatic, prostate, stomach, kidney, liver, melanocyte, muscle, ovarian, skin, testicular, or thyroid tumor. The cells may be from a cell line, which comprises substantially genetically identical cells. For example, the cells may be at least 90%, 95%, 96%, 97%, 98%, 99%, or more genetically identical. In some embodiments, the cells are descended from a single cell or from a single sample (e.g., a sample obtained from a tumor). In some embodiments, the populations of tumorigenic cells are substantially isogenic except with regard to the RNAi agent. Numerous tumorigenic cell lines are known in the art and may be used in various embodiments of the invention. For example, tumorigenic cell lines are available from sources such as the ATCC (or other repositories such as the DSMZ), the Karmanos Cancer Center (Michigan), and/or NIH or NCI (e.g., the NCI-60 cell cancer cell line panel). In some embodiments, the tumorigenic cells are engineered from non-tumorigenic somatic cells. For example, one or more oncogenes can be introduced into normal somatic cells to produce tumorigenic cells, as known in the art, and/or one or more tumor suppressor genes can be deleted, mutated, or otherwise inhibited. See, e.g., PCT/US2000/015008 (WO/2000/073420). In some embodiments, the tumorigenic cells are human cells. In some embodiments, the tumorigenic cells are genetically engineered to have at least one genetic alteration (such as deletion or mutation of a tumor suppressor gene or overexpression or activating mutation of an oncogene) in addition to harboring the RNAi agent. In many embodiments of the invention, the tumorigenic cells form a solid tumor in the animal host. In some embodiments, a cell population (e.g., cell line) is selected wherein a macroscopic tumor, e.g., a palpable tumor, reproducibly forms upon introduction of between 100 and 1,000,000 cells, e.g., between 10,000 and 1,000,000 cells, or between 100,000 and 1,000,000 cells into the animal host. In some embodiments, a palpable tumor is a tumor that can be felt by an investigator. In some embodiments, reproducibly means that an event occurs in at least 75%, 80%, 85%, or 90% of instances, under the same or substantially the same conditions. In some embodiments, cells are introduced together with a substance that may, for example, facilitate their engraftment in an organ. In some embodiments, cells are administered together with one or more extracellular matrix components or hydrogels. In some embodiments, Matrigel or collagen is used.

In some embodiments, the median or mean number of RNAi agents per cell genome is between about 0.5 and 2, e.g., about 1. In some embodiments, cells are infected with a virus harboring the RNAi agent at a MOI of about 0.5-1.0.

In some embodiments, the multiple populations of tumorigenic cells comprise at least 20, 50, 100, or more populations of cells (e.g., up to about 100, 200, 300, 400, or 500 populations of cells), each harboring in its genome an RNAi agent targeted to (i.e., having sequence correspondence to, and having the capacity to inhibit in a sequence-specific manner) a different gene. In some embodiments, the pool of cells comprises, for each of at least 5 different target genes, at least three populations of tumorigenic cells harboring different RNAi agents having sequence correspondence to the same target gene. Libraries of RNAi vectors are known in the art and can be used in embodiments of the present invention. RNAi agents can be selected to have sequence correspondence to unique sequences within the gene that they target (e.g., within a 5′ UTR, coding region, or 3′UTR). Multiple different RNAi agents targeted to the same gene can be used to reduce the likelihood that an “off-target” effect (i.e., inhibition of a gene other than the one that the RNAi agent is designed to inhibit) will be responsible for depletion of the RNAi agent. In some embodiments, at least 3, 4, or 5 RNAi agents per gene are used. In some embodiments, depletion of at least 2 distinct RNAi agents having sequence correspondence to a gene must occur in order for the gene to be considered a “hit” in a screen.

An animal host can be any non-human animal in various embodiments of the invention. In some embodiments of the invention, the animal host is a rodent, e.g., a rabbit, rat, or mouse. In some embodiments, the tumor cells are of a different species than the animal host. For example, the tumor cells can be human cells. In some embodiments, the animal host is immunocompromised. Immunocompromised animal hosts are known in the art. For example, the animal host may be selected or treated (e.g., with radiation or an immunosuppressive agent) to have a deficient immune system. In some embodiments, the animal host has a naturally occurring or engineered mutation that renders it immunodeficient. In some embodiments, the animal host is a SCID mouse, NOD mouse, NOD/SCID mouse, nude mouse, and/or Rag1 and/or Rag2 knockout mouse, or a rat having similar properties with respect to its immune system. In some embodiments, the immunocompromised animal substantially lacks T cells and/or B cells. In some embodiments, the animal host is a transgenic animal host. In some embodiments, the tumor cells are of the same species as the animal host. In some embodiments the tumor cells are substantially isogenic to the animal host.

In some embodiments, the tumorigenic cells are introduced at an orthotopic location (i.e., into an organ of the type from which the cells originate). For example, mammary tumor cells can be introduced into the mammary gland; prostate tumor cells can be introduced into the prostate gland; liver tumor cells can be introduced into the liver; lung tumor cells can be introduced into the lung, etc. In some embodiments, tumorigenic cells are introduced at a non-orthotopic location. In some embodiments, tumorigenic cells are introduced subcutaneously or under the renal capsule. In some embodiments, tumorigenic cells are introduced into the bloodstream. In some embodiments, a metastasis is harvested and, optionally, the abundance of RNAi agents contained therein is compared with the abundance of such agents in the introduced cells and/or in a primary tumor.

The target genes to be inhibited by the RNAi agents can be selected based on any criteria of interest to an investigator. In some embodiments, at least some of the distinct target genes are selected based at least in part on at least of the following properties: (i) higher expression in tumurs versus normal tissues, (ii) higher expression in aggressive cancer as compared with non-aggressive cancer of the same tissue; (iii) association with the stem cell state; (iv) subcellular localization; and (v) enzymatic activity. In some embodiments, for example, target genes comprise metabolic enzymes, transporters, kinases, receptors (e.g., growth factor receptors), genes that encode signaling molecules, genes that encode cytoplasmic, nuclear, transmembrane, or secreted proteins. In some embodiments, association with the stem cell state is assessed based on, e.g., expression of genes that are expressed selectively in stem cells (e.g., pluripotent or multipotent cells) or promoter occupancy by transcription factors at least partly specific to stem cells. In some embodiments a gene is considered to be associated with sternness if its average expression is greater than 4-fold upregulated in the stem versus differentiated cells profiles analyzed by Mikkelsen et al. and/or if its promoter is bound by at least two stem cell specific transcription factors (e.g., Oct4, Nanog, Sox2, Tcf3, Dax1, Nac1 or Klf4). In some embodiments, “higher expression” is expression that is higher than a value with which it is compared by at least about 1.5-, 2-, 5-, 10-, 20-fold, or more (e.g., up to about 100-fold, 200-fold, or more).

In some embodiments, RNAi agents are identified using DNA sequencing, e.g., massively parallel DNA sequencing, to determine the abundance of each RNAi agent in the genomic DNA of the tumor (or a sample thereof) and the initial pool of introduced cells. Massively parallel DNA sequencing can comprise use of Illumina sequencing technology or other high throughput (or “next generation”) sequencing approaches, which often entail performing large numbers, e.g., millions or billions of reads of short nucleic acids. In some embodiments, RNAi agents are identified by hybridization, e.g., to a nucleic acid array, e.g., an oligonucleotide array (oftern termed a “chip” in the art). Identifying an RNAi agent typically allows determination of which gene the RNAi agent inhibits. RNAi agents that are depleted in the tumor as compared with the introduced pool of cells, correspond to genes that are potential drug targets, for pharmacological agents to treat cancer.

In some embodiments, a compound is administered to the animal host prior to or during formation of the tumor. The compound, may, for example, be a candidate compound for treatment of cancer or a compound used in the art for treatment of cancer or a compound that promotes or enhances tumor formation or growth.

In some embodiments, a negative selection RNAi-based screen is performed in vitro. For example, tumorigenic cells harboring RNAi agents in their genome may be maintained in culture over a selectgled time period, after which RNAi agent abundance in the cultured cells is compared with abundance in the original population. RNAi agents that are depleted in the cell population after in vitro culture for a selected time period are identified. This approach allows, for example, identification of genes whose inhibition impairs survival and/or proliferation in vitro. In some embodiments, such genes also affect survival, proliferation, and/or tumor formation in vivo.

In some embodiments, a gene identified in an inventive screen encodes a protein that participates in a pathway (e.g., a signaling pathway, synthetic pathway) or biological process. The invention encompasses the recognition that genes involved in the same pathway or process as a gene identified in an inventive screen are also potential drug targets. A method of the invention may comprise determining whether a gene (or produce encoded thereby) identified in a screen is involved in a biological pathway or process and, if so, identifying other gene(s) (or products encoded thereby) as potential drug targets.

In some embodiments, a method further comprises (i) assessing the survival or proliferation of cells in which a gene identified in the screen, or a product encoded thereby, is inhibited. In some embodiments, the cells are tumorigenic cells, which may or may not be of the same type as the introduced cells. In some embodiments, a method further comprises: (I) assessing at least one property of tumorigenic cells or tumors in which a gene identified in the screen, or a product encoded thereby, is inhibited. For example, the property assessed can be tumor-initiating capacity, tumor growth rate, tumor size, tumor metastatis, tumor invasiveness, or response of the tumor cells or tumor to an agent (e.g, a therapeutic agent or candidate therapeutic agent) or condition. In some embodiments a method comprises comparing the survival or proliferation or a tumor-associated property of cells in which the gene or encoded product is inhibited with a reference value, e.g., a value obtained by assessing cells in which the gene, or its encoded product, is not inhibited or is inhibited to a different extent. In some embodiments, a gene is identified whose inhibition selectively inhibits survival or proliferation of tumor cells as compared with non-tumor cells.

In some aspects, the invention provides drug targets identified using an inventive in vivo RNAi-based screen. In some embodiments, a drug target for development of an anti-tumor agent is ABCE1, AMD1, AQP9, COX6B2, CTPS, CUBN, GAPDH, GLS2, GSTA4, HSD17B14, MTHFD2, PDE9A, PHGDH, PYCR1, SEPHS1, SLC15A1, SOD2, TPI1, TSTA3, or VDAC1, or a protein encoded by any of the foregoing genes. In some embodiments, a drug target for development of an anti-tumor agent is CTPS, GAPDH, GLS2, GMPS, NUDT5, PHGDH, PLA2G7, PYCR1, SEPHS1, SLC15A1, SLC16A3, SOD2, TALDO1, TPI1, TTYH3, or VDAC1, or a protein encoded by any of the foregoing genes. The invention encompasses performing compound screens to identify modulators (e.g., inhibitors or activators) of the identified drug targets. In some embodiments, an inhibitor of a drug target identified herein is a candidate agent for treatment of a tumor

II. Inhibiting the Serine Biosynthesis Pathway for Treatment of Cancer

In some aspects, the invention encompasses the identification of serine biosynthesis pathway components (e.g., enzymes involved in serine biosynthesis) as promising targets for treatment of cancer. As described in further detail in the Examples, through use of an inventive negative selection in vivo RNAi based screen, the gene that encodes phosphoglycerate dehydrogenase (PHGDH) was identified as a gene whose inhibition impairs survival and/or proliferation of tumorigenic cells. It was observed that PHGDH (the gene encoding PHGDH) is located in a genomic region of recurrent copy number gain in breast cancer, melanoma, and a variety of other cancer types including bone, esophageal, glioma, lung, chronic myelogenous leukemia (CML), meduloblastoma, neuroblastoma, ovarian, and soft tissue sarcoma. It is expected that overexpression of PHGDH via amplification and/or via other mechanisms is involved in promoting one or more aspects of tumorigenesis in additional tumor types.

PHGDH encodes 3-phosphoglycerate dehydrogenase, which is the first enzyme branching from glycolysis in the three-step pathway of serine biosynthesis (19) (FIG. 3a). PHGDH uses NAD as a cofactor to oxidize the glycolytic intermediate 3-phosphoglycerate into phospho-hydroxypyruvate (20, 21), which subsequent enzymes in the pathway convert into serine via transamination (PSAT1; Gene ID (Homo sapiens): 29968) and phosphate ester hydrolysis (PSPH; Gene ID (Homo sapiens): ID: 5723) reactions (19) (FIG. 3a). Serine is essential for protein synthesis and the synthesis of biomolecules needed for cell proliferation, including nucleotides, phosphatidyl-serine, and sphingosine (Supplementary FIG. 1).

Suppression of PHGDH in tumor cell lines that overexpress PHGDH was found to cause a dramatic decrease in cell proliferation. Short hairpin RNAs (shRNAs) that inhibit PHGDH expression inhibited tumor growth in an orthotopic model to degrees consistent with their capacity to suppress PHGDH expression. Moreover, tumors derived in vivo from cells that in culture had confirmed reductions in PHGDH levels had, in an immunohistochemical assay, PHGDH staining similar to control tumors, suggesting that tumorigenesis selected for cells that lost the shRNA-mediated suppression of PHGDH. These data further confirm the importance of PHGDH expression in tumor cell survival and/or proliferation and identify components of the serine biosynthesis pathway as drug targets for treatment of cancer. Furthermore, numerous genes that are expected to promote serine biosynthesis or are involved in the subsequent metabolism of serine for biosynthesis of other compounds were found to be elevated in ER-negative breast cancer, demonstrating that PHGDH elevation occurs in the context of upregulation of a broader pathway and identifying additional potential targets for discovery of agents useful for treating cancer (Supplementary FIG. 4).

In some aspects, the invention provides a method of treating a subject in need of treatment for a tumor, the method comprising administering a serine biosynthesis pathway inhibitor to the subject. In some embodiments, the tumor overexpresses at least one serine biosynthesis pathway enzyme (PHGDH, PSAT1, and/or PSPH). In some embodiments, the tumor exhibits PHGDH gene amplification. In some embodiments, the tumor exhibits overexpression of a SBP enzyme, e.g., PHGDH overexpression, as compared with normal tissue from the same organ or tissue as that from which the tumor arose or is believed to have arisen (or in the case of a tumor of unknown or undeterminable origin, a tissue an organ or tissue in which the tumor is located). In some embodiments, the tumor exhibits strong staining for PHGDH using an immunohistochemical assay. For example, the tumor may exhibit intense and reasonably uniform staining in at least 50% of the cells in a tissue section. In some embodiments, the tumor is an ER negative breast tumor. In some embodiments, the tumor exhibits PHGDH expression that would fall above the 20^{th}, 25^{th}, or 30^{th} percentile of staining intensities exhibited by ER negative breast tumors (i.e., within the 70%-80% of ER negative breast tumor that exhibit the greatest expression of PHGDH). In some embodiments, the tumor is a primary tumor and/or the tumor is not known to have metastasized. For example, in some embodiments the tumor is an ER negative breast tumor that has not metastasized, e.g., to bone. In some embodiments the tumor is an ER negative breast tumor that has not metastasized, e.g., to liver. In other embodiments, the tumor has detectably metastasized. In some embodiments, the tumor is a melanoma. In some embodiments, the tumor is a bone, esophageal, glioma, lung, chronic myelogenous leukemia (CML), meduloblastoma, neuroblastoma, ovarian, or soft tissue tumor.

In some embodiments, an inhibitor of a molecule or molecular complex could be any compound that, when contacted with a cell, results in decreased functional activity of the molecule or molecular complex, in the cell. In some embodiments, an inhibitor could be any compound that, when contacted with a molecule or molecular complex (e.g., an isolated molecule or molecular complex) decreases the activity of the molecule or complex. An inhibitor could act directly, e.g., by physically interacting with a molecule or complex to be inhibited, or indirectly such as by interacting with a different molecule or complex required for activity of the molecule or complex to be inhibited, or by interfering with expression or localization. A direct interaction could be a covalent binding or a non-covalent interaction. For example an irreversible inhibitor may bind covalently to an active site residue of an enzyme.

In some embodiments, a SBP inhibitor inhibits PHGDH, PSAT1, or PSPH. In some embodiments, a SBP inhibitor inhibits PHGDH. In some embodiments, serine biosynthesis inhibitor comprises a substrate analog or transition state analog. In some embodiments, a substrate analog is an analog of phosphoserine or an analog of phospho-hydroxypyruvate. In some embodiments, an analog is a non-hydrolyzable analog. Exemplary phosphoserine analogs include, e.g., sulfoserine, amino acid analogs containing a methylene substitution for the phosphate oxygen, 4-phosphono(difluoromethyl)phenylanaline, and L-2-amino-4-(phosphono)-4,4-difluorobutanoic acid. See, e.g., Otaka et al., Tetrahedron Letters 36:927-930 (1995). In some embodiments, a phosphoserine analog contains a non-hydrolyzable linkage to the phosphate group, e.g., a CF₂ group. See, e.g., U.S. Pat. No. 6,309,863

In some embodiments, a PSPH inhibitor is a compound described in: PHARMACEUTICAL COMPOSITION FOR INHIBITING PHOSPHOSERINE PHOSPHATASE ACTIVITY COMPRISING AN AMINO-TETRAHYDRO-BENZO[B]THIOPENE-3-CARBOXYLIC ACID DERIVATIVE. Korea patent 1020020033505. 2002; PHARMACEUTICAL COMPOSITION FOR INHIBITING PHOSPHOSERINE PHOSPHATASE ACTIVITY COMPRISING A BENZOQUINONE DERIVATIVE. Korea patent 1020020033506. 2002; or PHARMACEUTICAL COMPOSITION FOR INHIBITING PHOSPHOSERINE PHOSPHATASE ACTIVITY COMPRISING AN AMINOTHIOPENE CARBOXYLIC ACID DERIVATIVE. Korea patent 1020020033507. 2002.

The invention further provides methods of identifying tumors that overexpress PHGDH. In some aspects, such methods are of use to identify tumors that are likely to be responsive to therapy with an SBP inhibitor, e.g., a PHGDH inhibitor. In some aspects, the methods are of use to identify subjects who are candidates for therapy with an SBP inhibitor, e.g., a PHGDH inhibitor. For example, a subject having a tumor that overexpresses PHGDH is a candidate for treatment with an SBP inhibitor, e.g., a PHGDH inhibitor. In some embodiments, the subject has an ER negative breast cancer.

In some aspects, the invention provides an immunohistochemical (IHC) assay that permits reliable detection of PHGDH, e.g., in fixed tumor samples. As known in the art, IHC typically entails contacting a sample with an antibody (primary antibody) that binds to an entity (e.g., molecule or portion thereof) whose detection is of interest, allowing sufficient time for binding to occur, and detecting the antibody using any of a variety of different approaches. For example, the antibody can be recognized by a detectably labeled secondary antibody, which is then detected. In some aspects, the inventive IHC assay allows the assignment of tumors to different categories based on their PHGDH staining (see Examples). In some embodiments, tumors are classified as negative or positive for PHGDH staining, wherein a tumor that is negative for PHGDH staining is identified as not being likely to be responsive to an SBP inhibitor and/or a tumor that is positive for PHGDH staining (e.g., exhibits strong staining) has an increased likelihood of being responsive to therapy with a PHGDH inhibitor as compared with a tumor that is negative for PHGDH staining or exhibits moderate staining. In some embodiments, negative staining is poor or weak staining, e.g., undetectable or barely detectable staining. In some embodiments, strong staining is intense staining evident in at least 50%, or more of the cells visualized. In some embodiments, strong staining is within or about the average level of staining observed among the 70% of ER negative tumor that stain most strongly among a representative panel of ER negative breast tumors. See FIG. 2 for examples of strong, moderate, and weak PHGDH staining. In some embodiments, the sample comprises a tissue or cell sample, e.g., a surgical biopsy sample, a fine needle biopsy sample, cell brushing or washing, etc. In some embodiments, an alternate method of assessing PHGDH expression (e.g., Western blot) or mRNA measurement, is used to identify tumors that overexpress PHGDH.

In some embodiments, a sample is in the form of a fixed tissue section. In some embodiments a sample is in the form of fixed (e.g., acetone-fixed) cryostat section or fixed cell smear. The section may be a paraffin-embedded, formalin-fixed tissue section. The tissue section may be deparaffinized (i.e., much or all of the paraffin (or other substance in which the tissue section has been embedded) has been removed (at least sufficiently to allow staining of a portion of the tissue section) and the sample has been rehydrated. For example, deparaffinization and hydration may be performed in xylene and graded ethanol to distilled water. In some embodiments, a tissue section is subjected to an antigen retrieval procedure. A variety of antigen retrieval procedures can be used in embodiments of the IHC assay. In some embodiments, the antigen retrieval procedure entails exposing the sample to a temperature near or above boiling, e.g., between about 90° C. and about 125° C., for a period of time. In some embodiments, heat-induced epitope retrieval comprises immersion of tissue sections in a pre-heated buffer solution and maintaining heat in a water bath (e.g., 95-99° C.). Alternative heat sources may be used. In some embodiments, a pressure boiler is used. In some embodiments, heat is applied for about 30-60 minutes, e.g., about 40-45 minutes, after which the sample can be allowed to cool, e.g., to room temperature. In some embodiments, Target Retrieval Solution pH 9.0 (code 52368) or 10× Concentrate (code S2367), from Dako, is used according to manufacturer's instructions.

In some embodiments the method comprises performing IHC using a primary antibody that does not substantially react with mammalian proteins other than PHGDH. In some embodiments the method comprises performing IHC using antibody HPA021241 (available from Sigma) or an antibody that binds to at least one epitope to which antibody HPA021241 binds, on a sample obtained from the tumor. In some embodiments, the antibody that binds to at least one epitope in a polypeptide having the sequence LEEIWPLCDFITVHTPLLPSTTGLLNDNTFAQCKKGVRVVNCARGGIVDEGALLRAL QSGQCAGAALDVFTEEPPRDRALVDHENVISCPHLGASTKEAQSRCGEEIAVQFVDM (SEQ ID NO: 4). In some embodiments, the antibody is monoclonal. In some embodiments the antibody is polyclonal. As used herein, the term “antibody” refers to an immunoglobulin, whether natural or wholly or partially synthetically produced. An antibody may be a member of any immunoglobulin class, including any of the mammalian, e.g., human, classes: IgG, IgM, IgA, IgD, and IgE, or subclasses thereof, and may be an antibody fragment, in various embodiments of the invention. As used herein, the term “antibody fragment” refers to a derivative of an antibody which contains less than a complete antibody. In general, an antibody fragment retains at least a significant portion of the full-length antibody's specific binding ability. Examples of antibody fragments include, but are not limited to, Fab, Fab′, F(ab′)2, scFv, Fv, dsFv diabody, Fd fragments, and domain antibodies. Standard methods of antibody identification and production known in the art can be used to produce an antibody that binds to a polypeptide of interest. In some embodiments, an antibody is a monoclonal antibody. Monoclonal antibodies can be identified and produced, e.g., using hybridoma technology or recombinant nucleic acid technology (e.g., phage or yeast display). In some embodiments, an antibody is a chimeric or humanized antibody. In some embodiments a monoclonal antibody is a fully human antibody. Such antibodies can be identified, e.g., using a transgenic mouse comprising at least some unrearranged human immunoglobulin gene sequences and a disruption of endogenous heavy and light chain murine sequences or using display technology (e.g., phage or yeast display). See, e.g., Lonberg N. Fully human antibodies from transgenic mouse and phage display platforms. Curr Opin Immunol. 20(4):450-9, 2008. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may comprise a single chain antibody fragment. Alternatively or additionally, an antibody fragment may comprise multiple chains which are linked together, for example, by disulfide linkages. A functional antibody fragment typically comprises at least about 50 amino acids and more typically comprises at least about 100, e.g., about 200 amino acids. For example, an antibody fragment typically contains at least 1, 2, or 3 complementarity determining domains (CDRs) (VL CDR1, CDR2, CDR3; VH CDR1, CDR2, CDR3) of the antibody, optionally joined by one or more framework region(s). It will be appreciated that certain antibodies, e.g., recombinantly produced antibodies, can comprise heterologous sequences not derived from naturally occurring antibodies. For example, single-chain variable fragments (scFv) are typically fusion protein containing the variable regions of the heavy (VH) and light chains (VL) of immunoglobulins, connected with a short linker peptide of ten to about 25 amino acids. The linker is sometimes rich in glycine (e.g., for flexibility) and/or serine or threonine (e.g., for solubility), and can either connect the N-terminus of the VH with the C-terminus of the VL, or vice versa. Other heterologous sequences such as epitope tags (e.g., to facilitate purification) can be present.

In some aspects, the invention encompasses the recognition that PHGDH inhibition should be well tolerated in patients and would not require targeting or restricting the activity of a PHGDH inhibitor specifically to a tumor. Homozygous PHGDH loss-of-function mutations that result in little to no detectable PHGDH activity in humans and a knockout of PHGDH in mice have been described (29, 30). In both cases, loss of PHGDH activity causes low serine and glycine levels in the brain which affect neuronal function, but in humans this phenotype can been reversed by antenatal serine supplementation (31).

In some embodiments of the invention, a small molecule or other agent that targets PHGDH is selected, designed, and/or modified to not significantly cross the blood-brain barrier. Furthermore, because PHGDH suppression inhibits cell proliferation in the presence of serine (see Examples) and serine supplementation reverses the toxicity of the loss-of-function mutation, the present invention encompasses the recognition that serine supplementation can be used to mitigate any on-target toxicity that might occur due to administration of a serine biosynthesis inhibitor, e.g., a PHGDH inhibitor, while not interfering with the potential anti-tumor effects of the inhibitor. In some aspects, the invention provides a method of treating a subject in need of treatment for cancer, the method comprising administering a SBP inhibitor, e.g., a PHGDH inhibitor, to a subject in need of treatment for cancer, wherein the subject is not placed on a low serine diet or otherwise deprived of serine. In some aspects, the invention provides a method of treating a subject in need of treatment for cancer, the method comprising administering a SBP inhibitor, e.g., a PHGDH inhibitor, to a subject in need of treatment for cancer, wherein the subject also receives serine supplementation. For example, the subject may receive serine in amount at least 2, 5, or 10-fold the average amount of serine that the subject would otherwise consume (e.g., on a per day basis) and/or that would be recommended for consumption to maintain normal health.

In some embodiments, the invention provides methods of use for identifying or characterizing a SBP inhibitor and compositions of use in the methods. In some embodiments, the invention provides a composition comprising: (a) a 3-phosphoglycerate dehydrogenase (PHGDH) polypeptide; (b) a PHGDH substrate; and (c) a test agent. In some embodiments the invention provides methods of identifying and/or characterizing a PHGDH inhibitor using the afore-mentioned composition. In many embodiments, the components of the composition, e.g., the PHGDH polypeptide, are isolated components. In some aspects, “isolated” refers to a substance (e.g., cells, test agent, or other material) that is (i) separated from at least some other substances with which it is normally found in nature, usually by a process involving the hand of man, (ii) artificially produced (e.g., chemically or recombinantly synthesized), and/or (iii) present in an artificial environment or context (i.e., an environment or context in which it is not normally found in nature). In some embodiments, the PHGDH polypeptide is recombinantly produced and, optionally, comprises a tag. In some embodiments, the PHGDH polypeptide comprises a sequence identical to that of a naturally occurring PHGDH polypeptide, e.g., human PHGDH. In some embodiments, the polypeptide comprises a variant of a naturally occurring PHGDH polypeptide, e.g., a functional variant, that catalyzes the production of phospho-hydroxypyruvate from 3-phosphoglycerate in the composition in the absence of the test agent. For example, the variant may comprise a tag (e.g., an epitope tag) or may contain one or more amino acid alterations as compared with a naturally occurring polypeptide. A “variant” of a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments, the PHGDH substrate comprises 3-phosphoglycerate, and the composition optionally further comprises NAD+, and L-glutamine. In some embodiments, the composition farther comprises one or more components that serve as indicator(s) of PHGDH activity, e.g., one or more components that serve as indicator(s) of NADH production.

In some embodiments, the invention provides a method of identifying a candidate inhibitor or enhancer of PHGDH activity, the method comprising: (a) providing a composition comprising an PHGDH polypeptide, a PHGDH substrate, and a test agent; (b) determining whether presence of the test agent in the composition inhibits or enhances activity of PHGDH, wherein if presence of the test agent inhibits or enhances activity of PHGDH, the test agent is identified as a candidate inhibitor or enhancer of PHGDH, respectively; and (c) optionally confirming that a candidate inhibitor or enhancer of PHGDH activity is an inhibitor or enhancer of PHGDH activity (e.g., by repeating the assay). In some embodiments, an assay determines whether a test agent inhibits production of a product by an enzyme, e.g., PHGDH.

In some embodiments, the method of identifying a candidate anti-tumor agent, the method comprising: (a) providing a composition comprising an isolated PHGDH polypeptide, a PHGDH substrate, and a test agent; (b) determining whether presence of the test agent in the composition inhibits activity of PHGDH, wherein if presence of the test agent inhibits activity of PHGDH, the compound is identified as a candidate anti-tumor agent; and (c) optionally confirming that a candidate inhibitor of PHGDH activity is an inhibitor or enhancer of PHGDH activity (e.g., by repeating the assay), and/or or (c) optionally confirming that a candidate inhibitor of PHGDH activity has activity as an anti-tumor agent in vitro or in vivo.

A variety of rat tumors upregulate the activity of the serine synthesis pathway, as determined by enzyme assays in tumor lysates (19, 22), and suggest that PSPH is the rate-limiting enzyme in the pathway in the liver (23). In some embodiments such an enzyme assay is used to determine whether a test agent inhibits the SBP.

In some embodiments, an agent identified as a candidate inhibitor of the SBP is contacted with cells. In some embodiments, the cells comprise tumor cells. In some embodiments, effect of the compound on survival and/or proliferation of the cells and/or on serine biosynthesis pathway activity is assessed. Cells may be in living animal, e.g., a mammal, or may be isolated cells. Isolated cells may be primary cells, such as those recently isolated from an animal (e.g., cells that have undergone none or only a few population doublings and/or passages following isolation), or may be a cell of a cell line that is capable of prolonged proliferation in culture (e.g., for longer than 3 months) or indefinite proliferation) in culture (immortalized cells). In many embodiments, a cell is a somatic cell. Somatic cells may be obtained from an individual, e.g., a human, and cultured according to standard cell culture protocols known to those of ordinary skill in the art. Cells may be obtained from surgical specimens, tissue or cell biopsies, etc. In some embodiments, an isolated population of cells consists of at least 30%, 40%, 50%, 60%, 70%, 80%, 90%, 95%, 96%, 97%, 98%, 99%, or 100% cells of a particular cell type (i.e., the population is at least 30%, 40%, 50%, 60%, 70%, 80%, 90%, 95%, 96%, 97%, 98%, 99%, or 100% pure), e.g., as determined by expression of one or more markers or any other suitable method.

Any of a wide variety of agents (also termed “compounds”) can be tested to determine whether they inhibit the SBP, e.g., whether they inhibit PHGDH. Agents of use in various embodiments of the invention can comprise, e.g., small molecules, peptides, polypeptides, nucleic acids, oligonucleotides, etc. In some embodiments, an agent comprises two or more molecular entitities non-covalently associated with each other. Certain non-limiting examples of agents are discussed herein.

In some embodiments, an agent is a small molecule. A small molecule is often an organic compound having a molecular weight equal to or less than 2.0 kD, e.g., equal to or less than 1.5 kD, e.g., equal to or less than 1 kD, e.g., equal to or less than 500 daltons and usually multiple carbon-carbon bonds. Small molecules often comprise one or more functional groups that mediate structural interactions with proteins, e.g., hydrogen bonding, and typically include at least an amine, carbonyl, hydroxyl or carboxyl group, and in some embodiments at least two of the functional chemical groups. A small molecule may comprise cyclic carbon or heterocyclic structures and/or aromatic or polyaromatic structures substituted with one or more chemical functional groups and/or heteroatoms. In some embodiments a small molecule satisfies at least 3, 4, or all criteria of Lipinski's “Rule of Five”. In some embodiments, a compound is cell-permeable, e.g., within the range of typical compounds that act intracellularly, e.g., within mammalian cells. In some embodiments, the IC₅₀ or a compound, e.g., a small molecule, for a target to be inhibited is less than or equal to about 5 nM, 10 nM, 50 nM, 100 nM, 500 nM, 1 μM, 10 μM, 50 μM, or 100 μM.

In some embodiments a test agent comprises a nucleic acid, e.g., an oligonucleotide (which typically refers to short nucleic acids, e.g., 50 nucleotides in length or less), the invention contemplates use of oligonucleotides that are single-stranded, double-stranded (ds), blunt-ended, or double-stranded with overhangs, in various embodiments of the invention. Modifications (e.g., nucleoside and/or backbone modifications), non-standard nucleotides, delivery vehicles and systems, etc., known in the art as being useful in the context of siRNA or antisense-based molecules for research or therapeutic purposes is contemplated for use in various embodiments of the instant invention. siRNAs typically comprise two separate nucleic acid strands that are hybridized to each other to form a duplex. They can be synthesized in vitro, e.g., using standard nucleic acid synthesis techniques. A nucleic acid may contain one or more non-standard nucleotides, modified nucleosides (e.g., having modified bases and/or sugars) or nucleotide analogs, and/or have a modified backbone. Any modification or analog recognized in the art as being useful for RNAi, aptamers, antisense molecules or other uses of oligonucleotides can be used. Some modifications result in increased stability, cell uptake, potency, etc. Exemplary compound can comprise morpholinos or locked nucleic acids. In some embodiments the nucleic acid differs from standard RNA or DNA by having partial or complete 2′-O-methylation or 2′-O-methoxyethyl modification of sugar, phosphorothioate backbone, and/or a cholesterol-moiety at the 3′-end. In certain embodiments an siRNA or shRNA comprises a duplex about 19 nucleotides in length, wherein one or both strands has a 3′ overhang of 1-5 nucleotides in length (e.g., 2 nucleotides).

In some embodiments, a compound comprises a polypeptide. Polypeptides may contain any of the 20 amino acids that are naturally found in proteins and are genetically encoded (“standard” amino acids), other amino acids that are found in nature, and/or artificial amino acids or amino acid analogs. One or more of the amino acids in a polypeptide may be modified, for example, by the addition of a chemical entity such as a carbohydrate group, a phosphate group, a fatty acid group, an alkyl group etc.

Compounds can be produced using any suitable method known in the art. The skilled artisan will select an appropriate method based, e.g., on the nature of the compound. The production method can be partially or completely synthetic in various embodiments. In some embodiments a compound (or starting material for synthesis) is purified from an organism or other natural source, e.g., a plant, microbe, fermentation broth, etc. A compound of use in the invention may be provided as part of a composition, which may contain, e.g., an ion, salt, aqueous or non-aqueous diluent or carrier, buffer, preservative, etc, It is noted that although combined use of compounds is of particular interest, the use of compounds disclosed herein is not limited to their use in combination. In some embodiments of the invention,

In general, in any embodiment of the invention in which an SBP inhibitor, e.g., a PHGDH inhibitor is used, such inhibitor may be used at a concentration that inhibits one or more activities of its target by a selected amount and/or administered to a subject in an amount sufficient to achieve a selected reduction in activity in at least some cells of a tumor. For example, an amount may be sufficient to reduce activity in a sample obtained from a tumor by a selected amount. The activity may be reduced by at least, for example, about 5%, 10%, 15%, 20%, 25%, 30%, 35%, 40%, 45%, 50%, 55%, 60%, 65%, 70%, 75%, 80%, 85%, 90%, 95%, 99%, or 100% of a reference level (e.g., a control level). A reference level may be a level existing in the tumor prior to therapy or an average level existing in tumors that overexpress one or more SBP enzymes, e.g., tumors that exhibit strong staining for PHGDH.

As used herein, “inhibit”, or “inhibition” (and similar terms such as “reduce”, “reduction”, or “decrease”) may, or may not, be complete. For example, cell proliferation, also referred to as growth, may, or may not, be decreased to a state of complete arrest for an effect to be considered one of inhibition or reduction of cell proliferation. Similarly, enzyme activity or gene expression may, or may not, be decreased to a state of complete absence of activity or expression for an effect to be considered one of suppression, inhibition or reduction. Furthermore, “inhibition” may comprise preventing proliferation of a non-proliferating cell and/or inhibiting the proliferation of a proliferating cell. Similarly, inhibition of cell survival may refer to killing of a cell, or cells, such as by necrosis or apoptosis, and/or the process of rendering a cell susceptible to death. The suppression, inhibition, or reduction may be at least about 5%, 10%, 15%, 20%, 25%, 30%, 35%, 40%, 45%, 50%, 55%, 60%, 65%, 70%, 75%, 80%, 85%, 90%, 95%, 99% of a reference level (e.g., a control level). In some cases the level of inhibition or reduction compared with a control level is statistically significant. As used herein, “statistically significant” refers to a p-value of less than 0.05, e.g., a p-value of less than 0.025 or a p-value of less than 0.01, using an appropriate statistical test (e.g, ANOVA, t-test, etc.).

In certain embodiments, the survival and/or proliferation of a cell or cell population is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Other exemplary assays include BrdU, EdU, or H3-Thymidine incorporation assays; DNA content assays using a nucleic acid dye, such as Hoechst Dye, DAPI, Actinomycin D, 7-aminoactinomycin D or Propidium Iodide; Cellular metabolism assays such as AlamarBlue, MTT, XTT, and CellTitre Glo; Nuclear Fragmentation Assays; Cytoplasmic Histone Associated DNA Fragmentation Assay; PARP Cleavage Assay; TUNEL staining; and Annexin staining.

In some embodiments, a SBP inhibitor, e.g., a PHGDH inhibitor, is used to inhibit cell proliferation or survival in vitro, e.g., to assess the sensitivity of a subject's cells (e.g., tumor cells) to the inhibitor (or to a composition comprising the inhibitor and, optionally, one or more additional agent(s)). If the cells are sensitive, the compound may be administered to the subject. Cells can be contacted with compounds for various periods of time. In some embodiments cells are contacted for between 2 hours and 20 days, e.g., for between 6 hours and 10 days, for between 2 and 5 days, or any intervening range or particular value. Cells can be contacted transiently or continuously. If desired, a compound can be removed prior to assessing survival and/or proliferation (or other characteristics). In certain embodiments of any aspect of the invention, the cell is a vertebrate cell, e.g., a mammalian cell, e.g., a human cell. In certain embodiments the cell is a non-human animal cell, e.g., a rodent cell, e.g., mouse, rat, or rabbit cell. In certain embodiments the cell is one that proliferates aberrantly in a proliferative disease. In some embodiments the cell is a tumor cell. In some embodiments the tumor cell is a cancer stem cell. In certain embodiments the cell is a primary cell.

In some aspects, the invention relates to or makes use of genetically modified cells e.g., cells that have been genetically modified to render them tumorigenic. A “genetically modified” or “engineered” cell refers to a cell into which a nucleic acid has been introduced by a process involving the hand of man (or a descendant of such a cell that has inherited at least a portion of the nucleic acid). The nucleic acid may for example contain a sequence that is not naturally found in the cell, it may contain native sequences (i.e., sequences naturally found in the cell) but in a non-naturally occurring arrangement (e.g., a coding region linked to a promoter from a different gene), or altered versions of native sequences, etc. The process of transferring the nucleic acid into the cell can be achieved by any suitable technique and will often involve use of a vector. In some embodiments the nucleic acid or a portion thereof is integrated into the genome of the cell and/or is otherwise stably heritable. The nucleic acid may have subsequently been removed or excised from the genome, provided that such removal or excision results in a detectable alteration in the cell relative to an unmodified but otherwise equivalent cell. For example, the cell may have been engineered to overexpress an oncogene, to express a mutant version of an oncogene, and/or to have reduced or absent expression of a tumor suppressor gene.

In some embodiments the method comprises comparing the effect of an agent on a tumor cell or other aberrantly proliferating cell with the effect of such agent on a normal cell. In some embodiments the method comprises comparing the effect of an agent on a tumor with the effect on normal proliferating cells obtained from the same subject. In certain embodiments of the invention a compound displays selective activity (e.g., selective inhibition of survival and/or proliferation, selective toxicity) against target cells (e.g., abnormally proliferating cells or other undesired cells) relative to its activity against non-target cells (e.g., normal cells). In some embodiment, the 1050 of a compound may be at least about 2, 5, 10, 20, 50, 100, 250, 500, 1000, 10,000-fold or more lower for cancer cells than for non-cancer cells.

In some embodiments an agent is administered to a non-human subject, e.g., a non-human mammal, e.g., a rodent such as a mouse, rat, hamster, rabbit, or guinea pig; a dog, a cat, a bovine or ovine, a non-human primate, etc. In some embodiments, the subject may serve as an animal model useful for identifying, characterizing, and/or testing pharmacological agents, e.g., anti-cancer agents. For example, the subject may have a tumor xenograft or may be injected with tumor cells or have a predisposition to develop tumors at an abnormally high rate. In some embodiments the animal is immunocompromised. The non-human animal may be useful for assessing effect of an agent or composition on tumor formation, development, progression, metastasis, etc. In some embodiments the animal is used to assess efficacy and/or toxicity. Methods known in the art can be used for such assessment. In some embodiments, a compound is administered for veterinary purposes, e.g., to treat a vertebrate, e.g., domestic animal such as a dog, cat, horse, cow, sheep, etc. In some embodiments the animal is ovine, bovine, equine, feline, canine, or avian.

In some aspects, the invention relates to or makes use of genetically modified multi-cellular organisms. An organism at least some of whose cells are genetically engineered or that is derived from such a cell is considered a genetically engineered organism. Such an organism may be a non-human mammal. In some embodiments, the organism may serve as an animal model for cancer. For example, the subject may be a genetically engineered non-human mammal, e.g., a mouse, that has a predisposition to develop tumors. The mammal may overexpress an oncogene (e.g., as a transgene) or underexpress a tumor suppressor gene (e.g., the animal may have a mutation or deletion in the tumor suppressor gene).

In some aspects, a cell or organism is genetically modified using a suitable vector. As used herein, a “vector” may comprise any of a variety of nucleic acid molecules into which a desired nucleic acid may be inserted, e.g., by restriction digestion followed by ligation. A vector can be used for transport of such nucleic acid between different environments, e.g., to introduce the nucleic acid into a cell of interest and, optionally, to direct expression in such cell. Vectors are often composed of DNA although RNA vectors are also known. Vectors include, but are not limited to, plasmids and virus genomes or portions thereof. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics or other compounds, enzymes whose activities are detectable by standard assays known in the art (e.g., β-galactosidase or alkaline phosphatase), and proteins or RNAs that detectably affect the phenotype of transformed or transfected cells (e.g., fluorescent proteins). An expression vector is one into which a desired nucleic acid may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Regulatory elements may be contained in the vector or may be part of the inserted nucleic acid or inserted prior to or following insertion of the nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of skill in the art will be aware that the precise nature of the regulatory sequences needed for gene expression may vary between species or cell types, but can in general include, as necessary, 5′ non-transcribed and/or 5′ untranslated sequences that may be involved with the initiation of transcription and translation respectively, such as a TATA box, cap sequence, CAAT sequence, and the like. Other regulatory elements include IRES sequences. Such 5′ non-transcribed regulatory sequences will include a promoter region that includes a promoter sequence for transcriptional control of the operably linked gene. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. One of skill in the art is aware of regulatable (e.g., inducible or repressible) expression systems such as the Tet system and others that can be regulated by small molecules and the like, as well as tissue-specific and cell type specific regulatory elements. In some embodiments, a virus vector is selected from the group consisting of adenoviruses, adeno-associated viruses, poxviruses including vaccinia viruses and attenuated poxviruses, retroviruses (e.g., lentiviruses), Semliki Forest virus, Sindbis virus, etc. Optionally the virus is replication-defective. In some embodiments a replication-deficient retrovirus (i.e., a virus capable of directing synthesis of one or more desired transcripts, but incapable of manufacturing an infectious particle) is used. Various techniques may be employed for introducing nucleic acid molecules into cells. Such techniques include transfection of nucleic acid molecule-calcium phosphate precipitates, transfection of nucleic acid molecules associated with DEAR, transfection or infection with a virus that contains the nucleic acid molecule of interest, liposome-mediated transfection, nanoparticle-mediated transfection, and the like.

The invention encompasses testing a plurality of compounds, e.g., a compound library, to identify compound(s) that modulate, e.g., inhibit, a drug target (e.g., PHGDH). Compounds to be screened can come from any source, e.g., natural product libraries, combinatorial libraries, libraries of compounds that have been approved by the FDA or another health regulatory agency for use in treating humans, etc. The method may encompass performing high throughput screening. In some embodiments at least 100; 1,000; or 10,000 compounds are tested. Compounds identified as “hits” can then be tested in repeat assays and/or additional assays, e.g., to assess their effect on activity or expression of a drug target, cell proliferation or survival, tumor formation, growth, or metastatis, etc. Compounds identified as having a useful effect can be selected and systematically altered, e.g., using rational design, to optimize binding affinity, avidity, specificity, or other parameters. For example, one can screen a first library of compounds using the methods described herein, identify one or more compounds that are “hits” or “leads” (by virtue of, for example, their ability to inhibit metastasis), and subject those hits to systematic structural alteration to create a second library of compounds structurally related to the hit or lead. The second library can then be screened using the methods described herein or other methods known in the art. A compound can be modified or selected to achieve (i) improved potency, (ii) decreased toxicity and/or decreased side effects; (iii) modified onset of therapeutic action and/or duration of effect; and/or (iv) modified pharmacokinetic parameters (absorption, distribution, metabolism and/or excretion).

The invention provides methods of determining whether a compound that inhibits the SBP is suitable therapy for a subject in need of treatment, e.g., for a tumor. In some embodiments, cells are obtained from a subject in need of treatment and contacted with an SPB inhibitor in vitro. The ability of the compound to inhibit cell proliferation and/or survival is assessed. If the compound significantly inhibits cell proliferation and/or survival in concentrations correlating with those that are acceptable and achievable in vivo, the compound is a suitable therapy for the subject (or, said another way, the subject is a suitable candidate for treatment with the compound). Results of such an assay may be useful for selecting a therapeutic regimen for a subject, e.g., for selecting a dose and/or dosing schedule.

Compounds can be used or administered in a single dose or multiple doses, e.g., regularly for example, 1, 2, 3, or more times a day, weekly, bi-weekly, or monthly. In some embodiments, a compound is administered continuously to the subject (e.g., by release from an implant, pump, sustained release formulation, etc.). The dose administered can depend on multiple factors, including the identity of the compound, weight of the subject, frequency of administration, etc.

In certain embodiments, compositions and compound combinations of the present invention are provided for use in medicine, e.g., for treating a subject in need thereof. The subject may be suffering from a disease (e.g., a proliferative disease such as a cancer) warranting medical and/or surgical attention and/or may be at increased risk of developing a disease relative to an average member of the population and/or in need of prophylactic therapy. In certain embodiments, the compositions and/or methods are used in the treatment a disease characterized by abnormal, aberrant, or unwanted cell proliferation, e.g., cancer.

Exemplary tumors that may be treated using compounds of the present invention include colon cancer, lung cancer (e.g., small cell lung cancer, non-small cell lung cancer), bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, breast cancer, gastric cancer, eye cancer, gallbladder cancer, laryngeal cancer, oral cancer, penile cancer, glandular tumors, rectal cancer, small intestine cancer, gastrointestinal stromal tumors (GISTs), sarcoma, carcinoma, melanoma, urethral cancer, vaginal cancer, to name but a few.

In some embodiments, the cancer is a hematological malignancy. In some embodiments, the hematological malignancy is a lymphoma. In some embodiments, the hematological malignancy is a leukemia. Examples of hematological malignancies that may be treated using an inventive compound include, but are not limited to, acute lymphoblastic leukemia (ALL), acute myelogenous leukemia (AML), chronic myelogenous leukemia (CML), chronic lymphocytic leukemia (CLL), hairy cell leukemia, Hodgkin's lymphoma, non-Hodgkin's lymphoma, cutaneous T-cell lymphoma (CTCL), peripheral T-cell lymphoma (PTCL), Mantle cell lymphoma, B-cell lymphoma, acute lymphoblastic T cell leukemia (T-ALL), acute promyelocytic leukemia, and multiple myeloma.

In some embodiments, doses of compounds may range from about 1 μg to 10,000 mg, e.g., about 10 μg to 5000 mg, e.g., from about 100 μg to 1000 mg once or more per day, week, month, or other time interval. Stated in terms of subject body weight, doses in certain embodiments of the invention range from about 1 μg to 20 mg/kg/day, e.g., from about 1 m/kg/day to 10 mg/kg/day. In certain embodiments doses are expressed in terms of surface area rather than weight, e.g., between about 1 mg/m² to about 5,000 mg/m². The absolute amount will depend upon a variety of factors such as the concurrent treatment (if any), the number of doses and the individual patient parameters including age, physical condition, size and weight. These are factors well known to those of ordinary skill in the art and can be addressed with no more than routine experimentation. It is often the case that a maximum dose be used, that is, the highest safe dose according to sound medical judgment. In the case of compounds that have been tested already in preclinical studies and/or clinical trials, considerable information is already available that can be used in selecting doses.

As used herein, treatment or treating can include amelioration, cure, and/or maintenance of a cure (i.e., the prevention or delay of recurrence) of a disease, e.g., a proliferative disease, e.g., cancer. Treatment after a disorder has started aims to reduce, ameliorate or altogether eliminate the disorder, and/or at least some of its associated symptoms, to prevent it from becoming more severe, to slow the rate of progression, or to prevent the disorder from recurring once it has been initially eliminated. Treatment can be prophylactic, e.g., administered to a subject that has not been diagnosed with cancer, e.g., a subject with a significant risk of developing cancer. A subject at risk of cancer recurrence has been diagnosed with cancer and has been treated such that the cancer appears to be largely or completely eradicated. In some embodiments, a therapeutic method of the invention comprises providing a subject in need of treatment for a disease of interest herein, e.g., a proliferative disease, e.g., cancer. In some embodiments, a therapeutic method of the invention comprises diagnosing a subject in need of treatment for a disease of interest herein, e.g., cancer.

In some embodiments the subject is at risk of cancer or cancer recurrence. A subject at risk of cancer may be, e.g., a subject who has not been diagnosed with cancer but has an increased risk of developing cancer as compared with an age-matched control of the same sex. For example, the subject may have a risk at least 1.2 times that of an age and sex matched control. Determining whether a subject is considered “at risk” of cancer may be within the discretion of the skilled practitioner caring for the subject. Any suitable diagnostic test(s) and/or criteria can be used. For example, a subject may be considered “at risk” of developing cancer if (i) the subject has a mutation, genetic polymorphism, gene or protein expression profile, and/or presence of particular substances in the blood, associated with increased risk of developing or having cancer relative to other members of the general population not having such mutation or genetic polymorphism; (ii) the subject has one or more risk factors such as having a family history of cancer, having been exposed to a carcinogen or tumor-promoting agent or condition, e.g., asbestos, tobacco smoke, aflatoxin, radiation, chronic infection/inflammation, etc., advanced age; (iii) the subject has one or more symptoms or manifestations of cancer; (iv) the subject has been previously treated for cancer.

The compounds may be used in vitro or in vivo in an effective amount, by which is meant an amount sufficient to achieve a biological response of interest, e.g., reducing SBP activity, reducing cell proliferation or survival (e.g., reducing tumor cell proliferation or survival), reducing one or more symptoms or manifestations of a tumor and/or reducing the likelihood of recurrence or progression of a tumor.

Compounds, e.g., SBP inhibitors, may be administered in a pharmaceutical composition. A pharmaceutical composition can comprise a variety of pharmaceutically acceptable carriers. Pharmaceutically acceptable carriers are well known in the art and include, for example, aqueous solutions such as water, 5% dextrose, or physiologically buffered saline or other solvents or vehicles such as glycols, glycerol, oils such as olive oil or) injectable organic esters that are suitable for administration to a human or non-human subject. In some embodiments, a pharmaceutically acceptable carrier or composition is sterile. A pharmaceutical composition can comprise, in addition to the active agent, physiologically acceptable compounds that act, for example, as bulking agents, fillers, solubilizers, stabilizers, osmotic agents, uptake enhancers, etc. Physiologically acceptable compounds include, for example, carbohydrates, such as glucose, sucrose, lactose; dextrans; polyols such as mannitol; antioxidants, such as ascorbic acid or glutathione; preservatives; chelating agents; buffers; or other stabilizers or excipients. The choice of a pharmaceutically acceptable carrier(s) and/or physiologically acceptable compound(s) can depend for example, on the nature of the active agent, e.g., solubility, compatibility (meaning that the substances can be present together in the composition without interacting in a manner that would substantially reduce the pharmaceutical efficacy of the pharmaceutical composition under ordinary use situations) and/or route of administration of the composition. Compounds can be present as salts in a composition. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore it will be appreciated that agents may be modified, e.g., with targeting moieties, moieties that increase their uptake, biological half-life (e.g., pegylation), etc. It will be understood that compounds can exist in a variety or protonation states and can have a variety of configurations and may exist as solvates (e.g., with water (i.e. hydrates) or common solvents) or different crystalline forms (e.g., polymorphs). The structures presented here are intended to encompass embodiments exhibiting such alternative protonation states, configurations, and forms.

The pharmaceutical composition could be in the form of a liquid, gel, lotion, tablet, capsule, ointment, transdermal patch, etc. A pharmaceutical composition can be administered to a subject by various routes including, for example, parenteral administration. Exemplary routes of administration include intravenous administration; respiratory administration (e.g., by inhalation), nasal administration, intraperitoneal administration, oral administration, subcutaneous administration, intrasynovial administration, transdermal administration, and topical administration. For oral administration, the compounds can be formulated with pharmaceutically acceptable carriers as tablets, pills, dragees, capsules, liquids, gels, syrups, slurries, suspensions, etc. In some embodiments a compound may be administered directly to a tissue e.g., a tissue, e.g., in which cancer cells are or may be present or in which the cancer is likely to arise. Direct administration could be accomplished, e.g., by injection or by implanting a sustained release implant within the tissue. In some embodiments at least one of the compounds is administered by release from an implanted sustained release device, by osmotic pump or other drug delivery device. A sustained release implant could be implanted at any suitable site. In some embodiments, a sustained release implant may be particularly suitable for prophylactic treatment of subjects at risk of developing a recurrent cancer. In some embodiments, a sustained release implant delivers therapeutic levels of the active agent for at least 30 days, e.g., at least 60 days, e.g., up to 3 months, 6 months, or more. One skilled in the art would select an effective dose and administration regimen taking into consideration factors such as the patient's weight and general health, the particular condition being treated, etc. Exemplary doses may be selected using in vitro studies, tested in animal models, and/or in human clinical trials as standard in the art. If multiple compounds are administered, the compounds can be administered by the same or different routes (e.g., a first compound could be administered intravenously and a second compound administered orally).

In some embodiments, a pharmaceutical composition is delivered by means of a microparticle or nanoparticle or a liposome or other delivery vehicle or matrix. A number of biocompatible synthetic or naturally occurring polymeric materials are known in the art to be of use for drug delivery purposes. Examples include polylactide-co-glycolide, polycaprolactone, polyanhydride, cellulose derivatives, and copolymers or blends thereof. Liposomes, for example, which comprise phospholipids or other lipids, are relatively nontoxic, physiologically acceptable and metabolizable carriers that are relatively simple to make and administer. In some embodiments an agent is physically associated with a moiety that increases cell uptake, such as a cell-penetrating peptide, or a delivery agent. In some embodiments a delivery agent at least in part protects the compound from degradation, metabolism, or elimination from the body (e.g., increases the half-life). A variety of compositions and methods can be used to deliver agents to cells in vitro or in vivo. For example, compounds can be attached to a polyalkylene oxide, e.g., polyethylene glycol (PEG) or a derivative thereof, or incorporated into or attached to various types of molecules or particles such as liposomes, lipoplexes, or polymer-based particles, e.g., microparticles or nanoparticles composed at least in part of one or more biocompatible polymers or co-polymers comprising poly(lactide-glycolide), copolyoxalates, polycaprolactones, polyesteramides, polyorthoesters, polyhydroxybutyric acid, and/or polyanhydrides.

The invention provides pharmaceutical compositions comprising a SBP inhibitor. In some embodiments, the SBP inhibitor is a PHGDH inhibitor. The invention further provides a pharmaceutical composition comprising two or more SBP inhibitors. In some embodiments, the composition comprises inhibitors of at least two different SBP enzymes. The invention provides a pharmaceutical pack or kit containing a first pharmaceutical composition comprising a first SBP inhibitor and a second pharmaceutical composition comprising a second SBP inhibitor, wherein the SBP inhibitors are packaged in separate containers. A pharmaceutical composition may be in a container labeled with a label approved by a government agency responsible for regulating the manufacture, marketing, sale, and/or use of pharmaceutical agents and/or packaged with a package insert approved by such an agency that contains information relevant to the pharmaceutical composition, such as a description of its use in a method of the invention (e.g., instructions for use to treat cancer), contraindications, and/or potential side effects.

In some embodiments a compound (e.g., a SBP inhibitor) is formulated in unit dosage form, e.g., for ease of administration and uniformity of dosage. The term “unit dosage form” as used herein refers to a physically discrete unit of agent appropriate for the subject to be treated.

In some embodiments of the invention, a SBP inhibitor, e.g., a PHGDH inhibitor, is used together with one or more additional pharmacological therapies or non-pharmacological therapies (e.g., surgery, radiation), or combinations thereof, for treating a subject in need of treatment for a tumor. In some embodiments, a SBP inhibitor is administered in combination with one or more compounds selected from the group consisting of: trastuzumab (Herceptin), Cyclophosphamide, Epirubicin, Fluorouracil (5FU), Methotrexate, Mitomycin, Mitozantrone, Doxorubicin, Docetaxel (Taxotere), Gemcitabine (Gemzar).

Many cancer therapy regimens employ multiple chemotherapeutic agents in combination. See DeVita, cited above. Non-limiting examples of cancer chemotherapeutics that can be useful in some embodiments, with compounds and/or methods disclosed herein for treating cancer include alkylating and alkylating-like agents such as Nitrogen mustards (e.g., Chlorambucil, Chlormethine, Cyclophosphamide, Ifosfamide, and Melphalan), Nitrosoureas (e.g., Carmustine, Fotemustine, Lomustine, and Streptozocin), Platinum agents (i.e., alkylating-like agents) (e.g., Carboplatin, Cisplatin, Oxaliplatin, BBR3464, and Satraplatin), Busulfan, Dacarbazine, Procarbazine, Temozolomide, ThioTEPA, Treosulfan, and Uramustine; Antimetabolites such as Folk acids (e.g., Aminopterin, Methotrexate, Pemetrexed, and Raltitrexed); Purines such as Cladribine, Clofarabine, Fludarabine, Mercaptopurine, Pentostatin, and Thioguanine; Pyrimidines such as Capecitabine, Cytarabine, Fluorouracil, Floxuridine, and Gemcitabine; Spindle poisons/mitotic inhibitors such as Taxanes (e.g., Docetaxel, Paclitaxel) and Vincas (e.g., Vinblastine, Vincristine, Vindesine, and Vinorelbine); Cytotoxic/antitumor antibiotics such anthracyclines (e.g., Daunorubicin, Doxorubicin, Epirubicin, Idarubicin, Mitoxantrone, Pixantrone, and Valrubicin), compounds naturally produced by various species of Streptomyces (e.g., Actinomycin, Bleomycin, Mitomycin, Plicamycin) and Hydroxyurea; Topoisomerase inhibitors such as Camptotheca (e.g., Camptothecin, Topotecan and Irinotecan) and Podophyllums (e.g., Etoposide, Teniposide); monoclonal antibodies such as anti-receptor tyrosine kinases (e.g., Cetuximab, Panitumumab, Trastuzumab), anti-CD20 (e.g., Rituximab and Tositumomab), and others for example Alemtuzumab, Gemtuzumab; Photosensitizers such as Aminolevulinic acid, Methyl aminolevulinate, Porfimer sodium, and Verteporfin; Tyrosine kinase inhibitors such as Cediranib, Dasatinib, Erlotinib, Gefitinib, Imatinib, Lapatinib, Nilotinib, Sorafenib, Sunitinib, and Vandetanib; serine/threonine kinase inhibitors, (e.g., inhibitors of Abl, c-Kit, insulin receptor family member(s), EGF receptor family member(s), mTOR, Raf kinase family, phosphatidyl inositol (PI) kinases such as PI3 kinase, PI kinase-like kinase family members, cyclin dependent kinase family members, Aurora kinase family members), retinoids (e.g., Alitretinoin and Tretinoin), Hsp90 inhibitors, proteasome inhibitors (e.g., bortezomib), HDAC inhibitors, angiogenesis inhibitors, e.g., anti-vascular endothelial growth factor agents such as Bevacizumab (Avastin) or VEGF receptor antagonists, matrix metalloproteinase inhibitors, pro-apoptotic agents (e.g., apoptosis inducers), anti-inflammatory agents, etc.

In some embodiments, a SBP inhibitor is added to such a regimen or substituted for one or more of the compounds typically used in a combination chemotherapy regimen. Such combination therapies are an aspect of the invention. Some exemplary combinations of use, e.g., to treat breast cancer are:

CMF—cyclophosphamide, methotrexate and fluorouracil

FEC—epirubicin, cyclophosphamide and fluorouracil

FEC-T—epirubicin, cyclophosphamide, fluorouracil and taxotere

E-CMF—epirubicin, followed by CMF

AC—doxorubicin (adriamycin) and cyclophosphamide

EC—epirubicin and cyclophosphamide

MMM—methotrexate, mitozantrone and mitomycin

MM—methotrexate and mitozantrone.

In some embodiments, administration in combination of first and second agents (e.g., an SBP inhibitor and a second agent), is performed such that (i) a dose of the second agent is administered before more than 90% of the most recently administered dose of the first agent has been metabolized to an inactive form or excreted from the body; or (ii) doses of the first and second agent are administered within 4 weeks of each other (e.g., within 1, 2, 5, 7, 14, or 28 days of each other), or (iii) the agents are administered during overlapping time periods (e.g., by continuous or intermittent infusion); or (iv) any combination of the foregoing. In general, compounds can be administered in combination at appropriate time with respect to each other so as to achieve a desired effect greater than would be achieved using either agent alone. Multiple compounds are considered to be administered in combination if the afore-mentioned criteria are met with respect to all compounds, or in some embodiments, if each compound can be considered a “second compound” with respect to at least one other compound of the combination. The compounds may, but need not be, administered together as components of a single composition. In some embodiments, they may be administered individually at substantially the same time (e.g., within less than 1, 2, 5, or 10 minutes of one another). In some embodiments they may be administered individually within a short time of one another (by which is meant less than 3 hours, sometimes less than 1 hour, sometimes within 10 or 30 minutes apart). The compounds may, but need not, be administered by the same route of administration.

In certain embodiments in a combination therapy, use of a SBP inhibitor allows a reduction in dose of a second agent without reduction in efficacy. In certain embodiments at least one of the compounds (e.g., two or more compounds) is/are administered in an amount that would be sub-therapeutic or less than optimally therapeutic if the compound were administered as a single agent. A “sub-therapeutic amount” as used herein refers to an amount that is less than the amount that would produce a therapeutically useful result in the subject if administered in the absence of the other compound. In certain embodiments at least one of the compounds is administered in an amount that is lower than the maximum tolerated dose, e.g., the compound is administered in an amount that is about 5%, 10%, 20%, 30%, 40%, 50%, 60%, 70%, 80%, or 90% of the effective amount or maximum tolerated dose.

### EXAMPLES

### Example 1

### In Vivo RNAi Screen for Targets for Anticancer Drug Development

This Example describes development of an in vivo RNA i-based screening strategy to identify potential targets for anticancer drug development. Among other things, the screen permits identification of genes whose loss promotes tumorigenesis. Use of the screen to identify potential drug targets among genes that encode metabolic enzymes and transporters is also described in this Example.

As a starting point for identifying metabolic genes required for tumorigenesis, we cross-referenced maps of metabolic pathways with the KEGG database to compile a comprehensive list of 2,752 genes encoding all known human metabolic enzymes and transporters. Publicly available oncogenomic data were analyzed to score genes based on three properties: (i) higher expression in tumors versus normal tissues, (ii) high expression in aggressive breast cancer, or (iii) association with the stem cell state (FIG. 1a). Genes scoring in two of these three categories as well as those at the top of each category were selected to define a high priority set of 133 metabolic enzyme and transporter genes (Supplementary Table 2). (In Supplementary Table S2, 1=indicates genes that scored significantly in that category.) We assembled lentiviral shRNA vectors targeting these genes (median of 5 shRNAs per gene) and used them to generate two libraries of shRNA-expressing lentiviruses, one containing 235 distinct shRNAs (transporters plus control genes) and the other 516 distinct shRNAs (metabolic enzymes plus control genes). The lentiviral shRNA vectors from among which the libraries were assembled are described in reference (5).

To identify genes that may be essential for tumorigenesis, the libraries were screened for shRNAs that become depleted during breast tumor formation in mice. Human MCF10DCIS.COM cells (6) were chosen for the screens because, of several breast cancer lines examined, these were capable of forming tumors upon injection of the fewest number of cells. This property is desirable for purposes of undertaking negative selection screens involving hundreds of shRNAs, as the ability to measure changes in shRNA abundance is dependent upon those steps that introduce the greatest bottleneck into the pooled population. 1.5 million MCF10DCIS.COM cells were infected with each library so that each cell carried only one viral integrant, and ˜500-1000 cells per shRNA in the pool (100, 000-1 million cells total) were injected into mouse mammary fat pads at two sites per animal. Twenty-eight days later the resulting orthotopic tumors were harvested and massively parallel DNA sequencing was used to determine the abundance of each shRNA in the genomic DNA of the tumors and the initial pool of injected cells (FIG. 1b). shRNA abundances correlated well between replicate tumors (FIG. 1c), and 5 or 12 tumors per library were analyzed to identify the shRNAs that became significantly depleted during tumor formation. For 16 genes, at least 75% of the shRNAs targeting that gene scored and these genes were designated as hits in the screen (FIG. 1d).

Several genes previously shown to have important roles in cancer emerged as hits, including the mitochondrial ATP transporter VDAC1; the lactic acid transporter SLC16A3; and the nucleotide synthesis genes GMPS and CTPS (7,8,9), thus validating the ability of the negative selection approach to identify potential pharmacological targets for anticancer drug development. The hit list also includes genes involved in the control of oxidative stress (SOD2, GLS2, SEPHS1) (10,11,12), the pentose phosphate pathway (TALDO1) (13), glycolysis (GAPDH, TPI1), and in the proline (PYCR1) and serine (PHGDH) biosynthetic pathways, An analogous pooled screen carried out in MCF10ADCIS.com cells grown in culture rather than in tumor xenografts revealed that of the 20 genes that scored in the in vitro screen, 10 also scored in the in vivo screen (Supplementary FIG. 2b). Interestingly, AK2, which encodes an adenylate kinase that generates ADP, was required for in vitro but not in vivo growth (Supplementary FIG. 2c). Without wishing to be bound by any theory, one possible explanation for this finding is that nucleotide levels are much lower in tissue culture media than in blood.

### Example 2

### PHGDH Gene Amplification and mRNA Overexpression in Tumors

To prioritize the genes for follow up studies we asked if any were found to be amplified in a recently available analysis of copy number alteration across cancer genomes (14). Indeed, PHGDH exists in a region of chromosome 1p that is commonly amplified in breast cancer and melanoma (FIG. 2a), as well as in a number of other cancer types including bone, esophageal, glioma, lung, chronic myelogenous leukemia (CML), meduloblastoma, neuroblastoma, ovarian, and soft tissue sarcoma (data not shown). In total, 18% of patient derived breast cancer cell lines and 6% of primary tumors have amplifications in PHGDH. In the datasets examined, none of the other hit genes identified in our study are in genomic regions of focal and recurrent copy number gain.

We performed a meta analysis to investigate the association of PHGDH mRNA expression with various clinically significant features of breast cancer. We found that PHGDH mRNA levels are elevated in breast cancers that are ER-negative, of the basal type, and associated with poor 5-year survival (FIG. 2c) and additionally found that PHGDH is elevated in ER-negative breast cancer relative to normal breast tissue (FIG. 2b). Our identification of PHGDH in the meta analysis for genes associated with aggressive breast cancer is corroborated by another study which found elevated PHGDH mRNA levels in breast cancers that are ER-negative and associated with poor 5-year survival (15). Of all the genes identified as hits in our screen, PHGDH is the one with the most significantly elevated expression in ER-negative breast cancer (Supplementary FIG. 3).

### Example 3

### Knockdown of Genes Identified in the Screen Significantly Reduces Tumor Formation in Vivo

In knockdown-phenotype validation assays, the three PHGDH-targeting shRNAs that scored in the in vivo screen also decreased PHGDH protein expression (FIG. 1e). For subsequent validation studies, two shRNAs of differing knockdown efficacies were selected and, in the orthotopic tumor model, these shRNAs inhibited tumor growth to degrees consistent with their capacity to suppress PHGDH expression (FIG. 1e), For four additional genes that emerged from the in vivo screen (GMPS, SLC16A3, PYCR1, and VDAC1), two shRNAs that scored for each gene were tested for their effects on tumor formation in vivo. Introduction of these shRNAs into MCF10ADCIS.com cells suppressed expression of the expected targets and caused, compared to a control shRNA, a significant reduction in the capacity of the cells to form tumors (Supplementary FIG. 2a),

### Example 4

### Immunohistochemical Assay for PHGDH Protein Expression

We developed an immunohistochemical assay suitable for measuring PHGDH protein expression in fixed tissue samples. Using this assay, we analyzed 80 human breast tumor samples and found that PHGDH protein levels correlate significantly with ER-negative status (FIG. 2d). In total, compared to ER-positive breast tumors, ˜68% and ˜70% of ER-negative breast tumors have elevations of PHGDH at the mRNA and protein levels, respectively. ER-negative breast cancer comprises approximately 20-25% of all breast cancer cases, but as many as 50% of all breast cancer deaths within 5 years of diagnosis (16), underscoring the importance of identifying additional drug targets for this class of breast cancer.

Across a selected set of breast cancer lines, four lines with PHGDH amplifications had 8-12 fold higher PHGDH protein expression than the five lines without amplifications (FIG. 2e). Mechanisms other than gene copy number increases must also exist for boosting PHGDH expression because PHGDH protein levels were also elevated (FIG. 20 in two ER-negative cell lines (MT3, Hs578T) lacking the PHGDH amplification. This is consistent with the finding that PHGDH expression is upregulated at the mRNA and protein level in a higher fraction of ER-negative breast cancers than the fraction exhibiting amplification at the DNA level. Interestingly, PHGDH is also expressed 4-fold more in the MCF10DCIS.COM cells used in the in vivo screen than in two parental lines (MCF-10A; MCF10AT) that exhibit no or lower tumorigenicity (17) (FIG. 2g).

We further found that numerous genes that are expected to promote serine biosynthesis or are involved in the subsequent metabolism of serine for biosynthesis of various compounds are elevated in ER-negative breast cancer (Supplementary FIG. 4), demonstrating that PHGDH elevation occurs in the context of upregulation of a broader pathway.

### Example 5

### Validation of PHGDH as a Target for Anticancer Drug Development in Tumors that Overexpress PHGDH

We investigated whether cells with an increase in PHGDH expression require it for cell proliferation and survival. In cell lines with (BT-20, MDA-MB-468, HCC70, Hs578T and MT3), but not without (MDA-M13-231, MCF-7), elevated PHGDH expression, RNAi-mediated suppression of PHGDH caused a dramatic decrease in cell number (FIG. 3e and Supplementary FIG. 5b) and cell morphological changes suggestive of cell lethality (FIG. 3f) in the absence of apoptotic markers (Supplementary FIG. 5a). The sensitivity to PHGDH suppression was observed both in cells with PHGDH amplifications (BT-20, MDA-MB-468, HCC70) and in those with high PHGDII expression but lacking the amplifications (MT3, Hs578T). Suppression of the other two enzymes in the pathway (PSAT1 and PSPH) also inhibited the proliferation of MDA-MB-468 and BT-20 but not MCF7 cells (FIG. 3g). Therefore, elevated PHGDH expression defines a set of breast cancer cell lines that are dependent upon PHGDH, PSAT1, and PSPH for their proliferation. This finding suggests that the many ER-negative breast cancers that express PHGDH at high levels (˜70% of all ER-negative disease in our dataset) may be sensitive to inhibitors of the serine synthesis pathway.

In MCF10DCIS.com cells, suppression of PHGDH prior to xenografting of the cells decreased their tumor-forming capacity (FIG. 1e). To investigate whether suppression of PHGDH can also affect the growth of established tumors, we generated a doxycyline-inducible shRNA that, upon doxycycline treatment, effectively reduced PHGDH protein levels in MDA-MB-468 cells (FIG. 3h). MDA-MB-468 cells transduced with the inducible shRNA, but not treated with doxycycline, were injected into the murine mammary fat pad of immunocompromised mice and allowed to form tumors, which became palpable after 25 days. Mice were then given water with or without doxycycline and tumor size was monitored for the next 43 days. Compared to control mice, in mice given doxycycline tumor growth was substantially reduced (FIG. 3h). Tumors made with cells transduced with a control shRNA grew the same in the presence or absence of doxycycline (FIG. 3h). These results indicate that PHGDH suppression can adversely affect tumor growth in vivo.

We also found that PHGDH suppression inhibited proliferation even in cells growing in media containing normal levels of extracellular serine (FIG. 3e), and the supplementation of the media with additional serine or a cell-permeable methyl-serine-ester did not blunt the effects of the PHGDH knockdown (FIG. 4a, 4b). Without wishing to be bound by any theory, these results suggest that serine production per se may not represent the important role of PHGDH in promoting proliferation of tumor cell lines with high PHGDH expression.

Our work provides evidence that targeting the serine synthesis pathway will be therapeutically valuable in breast cancers (and other cancers) with elevated PHGDH expression due, e.g., to PHGDH amplifications and/or other mechanisms. As we find that ˜70% of ER-negative breast cancers exhibit elevated PHGDH, inhibition of the serine synthesis pathway has broad applicability in this subset of breast tumors.

### REFERENCES

¹Jones, R. G. & Thompson, C. B., Tumor suppressors and cell metabolism: a recipe for cancer growth. Genes Dev 23 (5), 537-548 (2009).
²Hsu, P. P. & Sabatini, D. M., Cancer cell metabolism: Warburg and beyond. Cell 134 (5), 703-707 (2008).
Tennant, D. A., Duran, R. V., & Gottlieb, E., Targeting metabolic transformation for cancer therapy. Nat Rev Cancer 10 (4), 267-277.
⁴Bric, A. et al., Functional identification of tumor-suppressor genes through an in vivo RNA interference screen in a mouse lymphoma model. Cancer Cell 16 (4), 324-335 (2009).
⁵Moffat, J. et al., A lentiviral RNAi library for human and mouse genes applied to an arrayed viral high-content screen. Cell 124 (6), 1283-1298 (2006).
⁶Miller, F. R., Santner, S. J., Tait, L., & Dawson, P. J., MCF10DCIS.com xenograft model of human comedo ductal carcinoma in situ. J Natl Cancer Inst 92 (14), 1185-1186 (2000).
⁷Simura, E., Shimada, H., Hatta, T., & Hirai, K., Mitochondrial voltage-dependent anion channels (VDACs) as novel pharmacological targets for anti-cancer agents. J Bioenerg Biomembr 40 (3), 213-217 (2008).
Ganapathy, V., Thangaraju, M., & Prasad, P. D., Nutrient transporters in cancer: relevance to Warburg hypothesis and beyond. Pharmacal Ther 121 (1), 29-40 (2009).
⁹Salvatore, G. at al., A cell proliferation and chromosomal instability signature in anaplastic thyroid carcinoma. Cancer Res 67 (21), 10148-10158 (2007).
¹⁰Weisiger, R. A. & Fridovich, I., Superoxide dismutase. Organelle specificity. J Biol Chem 248 (10), 3582-3592 (1973).
¹¹Hu, W. at al., Glutaminase 2, a novel p53 target gene regulating energy metabolism and antioxidant function. Proc Natl Acad Sci USA 107 (16), 7455-7460.
¹²Reeves, M. A. & Hoffmann, P. R., The human selenoproteome: recent insights into functions and regulation. Cell Mol Life Sci 66 (15), 2457-2478 (2009).
¹³Samland, A. K. & Sprenger, G. A., Transaldolase: from biochemistry to human disease. Int J Biochem Cell Biol 41 (7), 1482-1494 (2009).
¹⁴Beroukhim, R. et al., The landscape of somatic copy-number alteration across human cancers. Nature 463 (7283), 899-905.
¹⁵Pollari, S. et al., Enhanced serine production by bone metastatic breast cancer cells stimulates osteoclastogenesis. Breast Cancer Res Treat.
¹⁶van de Vijver, M. J. at al., A gene-expression signature as a predictor of survival in breast cancer. N Engl J Med 347 (25), 1999-2009 (2002).
¹⁷Miller, F. R. et al., Xenograft model of progressive human proliferative breast disease. J Natl Cancer Inst 85 (21), 1725-1732 (1993).
¹⁸Hu, M. et al., Regulation of in situ to invasive breast carcinoma transition. Cancer Cell 13 (5), 394-406 (2008).
¹⁹Snell, K., Enzymes of serine metabolism in normal, developing and neoplastic rat tissues. Adv Enzyme Regal 22, 325-400 (1984).
²⁰Achouri, Y., Rider, M. H., Schaftingen, & Robbi, M., Cloning, sequencing and expression of rat liver 3-phosphoglycerate dehydrogenase. Biochem J 323 (Pt 2), 365-370 (1997).
²¹Walsh, D. A. & Sallach, H.J., Purification and properties of chicken liver D-3-phosphoglycerate dehydrogenase. Biochemistry 4 (6), 1076-1085 (1965).
²²Knox, W. E., Herzfeld, A., & Hudson, J., Phosphoserine phosphatase distribution in normal and neoplastic rat tissues. Arch Biochem Biophys 132 (2), 397-403 (1969).
²³Lund, K., Merrill, D. K., & Guynn, R. W., The reactions of the phosphorylated pathway of L-serine biosynthesis: thermodynamic relationships in rabbit liver in vivo. Arch Biochem Biophys 237 (1), 186-196 (1985).
²⁴Vander Heiden, M. G., Cantley, L. C., & Thompson, C. B., Understanding the Warburg effect: the metabolic requirements of cell proliferation. Science 324 (5930), 1029-1033 (2009).
²⁵DeBerardinis, Lum, J. J., Hatzivassiliou, G., & Thompson, C. B., The biology of cancer: metabolic reprogramming fuels cell growth and proliferation. Cell Metab 7 (1), 11-20 (2008).
²⁶DeBerardinis, R. J. et al., Beyond aerobic glycolysis: transformed cells can engage in glutamine metabolism that exceeds the requirement for protein and nucleotide synthesis. Proc Natl Acad Sci USA 104 (49), 19345-19350 (2007).
²⁷Yun, J. et al., Glucose deprivation contributes to the development of KRAS pathway mutations in tumor cells. Science 325 (5947), 1555-1559 (2009).
²⁸Dang, C. V. et al., The c-Myc target gene network. Semin Cancer Biol 16 (4), 253-264 (2006).
²⁹Tabatabaie, L. et al., Novel mutations in 3-phosphoglycerate dehydrogenase (PHGDH) are distributed throughout the protein and result in altered enzyme kinetics. Hum Mutat 30 (5), 749-756 (2009).
³⁰Yoshida, K. et al., Targeted disruption of the mouse 3-phosphoglycerate dehydrogenase gene causes severe neurodevelopmental defects and results in embryonic lethality. J Biol Chem 279 (5), 3573-3577 (2004).
³¹de Koning, T. J. et al., Prenatal and early postnatal treatment in 3-phosphoglycerate-dehydrogenase deficiency. Lancet 364 (9452), 2221-2222 (2004).

Materials.

Materials were obtained from the following sources: antibodies to PHGDH (HPA021241) and PSPH(HPA020376) from Sigma; an antibody against PYCR1 (13108-1-AP) from Proteintech; an antibody against GMPS (A302-417A) from Bethyl Labs; an antibody against VDAC1 (ab16814) from Abeam; an antibody to RPS6 (2217), PARP (9532) and Caspase-3 (9662) from Cell Signaling Technologies; an antibody against PSAT1 (H00029968-A01) from Novus Biologicals; an antibody against SLC16A3 (AB3316P) from Millipore; and HRP-conjugated anti-mouse, anti-rabbit secondary antibodies from Santa-Cruz Biotechnology; MT-3 cells from DSMZ; Hs578T, MDA-MB-468, MDA-MB-231, BT-20, HCC1599, HCC70, DU4475, MCF-7 and ZR-75-30 cells from ATCC; MCF-10A, MCF-10AT1 and MCF10DCIS.com cells from the Karmanos Cancer Center, Michigan; matrigel from BD Biosciences; Phusion DNA polymerase from New England Biolabs; BCA Protein Assay from Pierce; and amino acid-free, glucose-free RPMI-1640 from US Biological. Lentiviral shRNAs targeting GFP as well as human PHGDH and PSPH were obtained from the The RNAi Consortium (TRC) collection of the Broad Institute (1). The TRC#s for the shRNAs used are: GFP, TRCN0000072186; PHGDH_{—}1, TRCN0000221861; PHGDH_{—}2, TRCN0000221865; PSPH_{—}1, TRCN0000002796; PSPH1_{—}2, TRCN0000315168; PSAT1_{—}1, TRCN0000035266; PSAT1_{—}2, TRCN0000035268; SLC16A3_{—}1, TRCN0000038477; SLC16A3_{—}2, TRCN0000038478; VDAC1_{—}1, TRCN0000029126; VDAC1_{—}2, TRCN0000029127; GMPS_{—}1, TRCN0000045938; GMPS_{—}2, TRCN0000045941; PYCR1_{—}1, TRCN0000038979; PYCR1_{—}2, TRCN0000038980. The TRC website is: http://www.broadinstitute.org/rnai/trc/lib

Methods.

Cell Culture.

MDA-MB-468, MDA-MB-231, BT-20, HCC1599, HCC70, DU4475, ZR-75-30, MT-3, Hs578T and MCF-7 were cultured in RPMI supplemented with 10% IFS and penicillin/streptomycin. MCF-10A and MCF10AT1 cells were cultured as described previously (2). MCF10DCIS.com cells were cultured in 50:50 DMEM and F12 media with 5% horse serum and penicillin/streptomycin.

Compilation of Metabolic Gene List.

A list of all human metabolic enzymes and small molecule transporters were generated by cross-referencing maps of metabolic pathways (Roche) with the KEGG database (http://www.genome.jp/kegg/kegg1.html). NCBI resources including Entrez Gene (http://www.ncbi.nlm.nih.gov/gene) and the available literature were used to identify known or putative gene function and to identify functional homologs. A gene was considered a metabolic enzyme if it modified a small molecule to generate another small molecule. Genes which modified polymerized DNA or RNA or which modified proteins were excluded. In cases where an enzyme could modify both a small molecule and a macromolecule, we favored a more liberal criterion of inclusion. A gene was considered a small molecule transporter if it formed a pore or channel through which a small molecule could traverse a lipid bilayer. Accessory or regulatory subunits of larger protein complexes were generally excluded.

Meta-Analysis of Oncogenomic Data.

To generate a cancer-relevant ‘high priority’ subset of metabolic genes (out of the 2,752 genes we classified as metabolic enzymes or small molecule transporters), we first identified those genes whose expression is significantly associated with the transformed state, advanced breast cancer, or sternness. Genes associated with the transformed state were obtained by analyzing 36 gene expression studies deposited in Oncomine (3) that profiled normal human tissue and primary tumors derived from them. The gene expression profiles in each study were classified as normal or tumor and for each group the log 2 median centered intensity for each gene was determined. A p-value associated with the significance of the difference between the two groups was calculated with the student t-test. After ranking the genes based on the p-values, the top 10% of the genes with lowest p-values were selected from each of the 36 studies. From these genes we identified those that are in the top 10% of the most upregulated metabolic genes across the all 36 studies at a p-value <0.05. Genes associated with aggressive breast cancer were obtained by analyzing 15 gene expression studies from Oncomine that profiled ER-negative versus ER-positive tumors, Grade 3 versus Grade 1 or 2 tumors, tumors of basal versus epithelial morphology, or tumors from patients who failed to survive after 5 years of follow-up versus those who did survived at 5 years. The 15 studies were analyzed as above to identify those genes which are in the top 10% of the most upregulated metabolic genes across the studies at a p-value <0.05. To identify genes associated with sternness, we analyzed gene expression studies comparing differentiated cells with stem cells (4), chromatin immunoprecipitation studies of stem cell-associated transcription factors (5, 6), and a previous meta-analysis of sternness-associated genes (7). Genes were considered to be associated with sternness if their average expression was greater than 4-fold upregulated in the stem versus differentiated cells profiles analyzed by Mikkelsen et al. (4) or if their promoters were bound by at least two stem cell specific transcription factors (Oct4, Nanog, Sox2, Tcf3, Dax1, Nac1 or Klf4) in both studies analyzed. To generate the final high priority set of 133 genes that was screened (Supplementary Table 2), three categories of genes were selected: (1) genes scoring in all three analyses, (2) the most significantly scoring ˜5% of genes in any one category, and (3) the most significantly scoring ˜10% of genes in any two categories.

Identification of Cell Lines for Use in Pooled Screening

In order to undertake negative selection RNAi screening, a cell line which could form a tumor upon injection of the minimum number of cells was identified. To accomplish this, 11 breast cell lines which previously identified as capable of forming tumors were selected and 100,000 cells from each were injected into the 4^{th} murine mammary fat pad. The cell lines tested included BT-20, BT-474, MCF10DCIS.com, HBL100, MCF7, MDA-MB-157, MDA-MB-231, MDA-MB-361, MDA-MB-453, T47D, and ZR-75-1. After one month, tumors were scored by size and number scoring per site, and tumors or injection sites were analyzed histologically to verify the presence of a tumor, or to identify microscopic tumors. In the timeframe of the experiment, MDA-MB-231, MDA-MB-361, MDA-MB-453, MCF7 and T47D cells formed microscopic tumors, whereas MCF10DCIS.com formed large tumors and ZR-75-1 formed small macroscopic tumors reproducibly. MCF10DCIS.com cells were then injected into murine mammary fat pads at 100,000, 10,000, 1,000 and 100 cells per site. All of these injections were capable of forming tumors, and tumor size correlated with the number of cells injected. The MCF10DCIS.com cell line was shown to be suitable for in vivo screening upon performing a screen using 180 shRNAs and demonstrating that nearly all of the shRNAs introduced initially could be recovered from the tumor and that replicate tumors exhibited significant correlation in those shRNAs over or under-represented compared to the injected pool. These experiments should not be construed to indicate that the other cell lines would not also be suitable for in vivo screening, as they were not tested using an shRNA pool (such testing is also not a requirement).

Pooled shRNA Screening

pLKO.1 lentiviral plasmids encoding shRNAs targeting the 133 transporters and metabolic enzymes listed in Supplementary Table 2 were obtained and combined to generate two plasmid pools. One contained the plasmids encoding shRNAs targeting all 47 transporters and another the plasmids encoding shRNAs targeting all 86 metabolic enzymes as well as control shRNAs designed not to target any gene. These plasmid pools were used to generate lentivirus-containing supernatants as described (8). MCF10DCIS.com cells were infected with the pooled virus so as to ensure that each cell contained only one viral integrant. Cells were selected for 3 days with 0.5 ug/mL puromycin. For the in vivo screen, cells were injected in 33% growth factor reduced matrigel into the fourth mammary fat pad of NOD.CB17 Scid/J mice (Jackson Labs) at 100,000 to 1,000,000 cells per injection site and tumors were harvested 4 weeks after implantation. For the in vitro screen, cells were plated in replicates of four at 1,000,000 per 10 cm plate and split at 1:8 once confluent (every 3-5 days) for 25-28 days. Genomic DNA was isolated from tumors or cells by digestion with proteinase K followed by isopropanol precipitation. To amplify the shRNAs encoded in the genomic DNA, PCR was performed for 33 cycles at an annealing temperature of 66° C. using 2-6 ug of genomic DNA, the primer pair indicated below, and DNA polymerase. So that PCR products obtained from many different tumors could be sequenced together, forward primers containing unique 2-nucleotide barcodes were used (see below). After purification, the PCR products from each tumor were quantified by ethidium bromide staining after gel electrophoresis, pooled at equal proportions, and analyzed by high throughput sequencing (Illumina) using the primer indicated below. shRNAs from up to 16 genomic DNA samples were sequenced together. Sequencing reads were deconvoluted using GNU Octave software by segregating the sequencing data by barcode and matching the shRNA stem sequences to those expected to be present in the shRNA pool, allowing for mismatches of up to 3 nucleotides. The Log 2 values reported are the average Log base 2 of the fold change in the abundance of each shRNA in the pre-injection cells compared to tumors for n=5 tumors for the transporter pool and n=12 tumors metabolic enzyme pool or to cells at day 25-28 for n=4 in vitro cultures. P-values were determined by two-sided homoscedastic unpaired t-test comparing each shRNA to a basket of negative control shRNAs contained within the shRNA pools. Individual shRNAs were identified as scoring in the screens using a p-value cutoff of 0.05 and Log 2 fold change cutoff of −1. Genes for which >75% of the shRNAs targeting the gene scored were considered hits. Individual shRNAs were considered to be differentially required in vitro versus in vivo using a p-value cutoff of 0.05 by a two-sided homoscedastic unpaired t-test comparing the in vitro and in vivo shRNA Log 2 fold change scores. For the transporter pool screen, this required normalization to the median of the two distributions. shRNAs present at less than 30 reads in the pre-injection cell sample were eliminated from further analysis. All experiments involving mice were carried out with approval from the Committee for Animal Care at MIT and under supervision of the Department of Comparative Medicine at MIT.

Primers for Amplifying shRNAs Encoded in Genomic DNA:

Barcoded Forward Primer (‘N’s indicate location of
sample-specific barcode sequence):
(SEQ ID NO: 1)
AATGATACGGCGACCACCGAGAAAGTATTTCGATTTCTTGGCTTTATAT
ATCTTGTGGAANNGACGAAAC
Common Reverse Primer:
(SEQ ID NO: 2)
CAAGCAGAAGACGGCATACGAGCTCTTCCGATCTTGTGGATGAATACT
GCCATTTGTCTCGAGGTC
Illumina Sequencing Primer:
(SEQ ID NO: 3)
AGTATTTCGATTTCTTGGCTTTATATATCTTGTGGAA

Analysis of Gene Copy Number Data

The significance of copy number alteration across multiple data sets was determined using the GISTIC algorithm with methods described in (9) and using the data deposited at http://www.broadinstitute.org/tumorscape.

Determination of Proportion of Tumors with PHGDH Over-Expression

To determine the percentage of breast cancers with elevations in PHGDH mRNA levels, data deposited in Oncomine from van de Vijver et al (10) was utilized. An ER-negative tumor was considered to have elevated PHGDH mRNA if the expression level was higher than 1.5SD above the mean expression level in the ER-positive class (˜91^{st} percentile). For the percentage of breast cancer exhibiting elevated PHGDH protein, data reported in FIG. 2c was utilized. An ER-negative tumor was considered to have elevated PHGDH protein if the immunohistochemical staining signal was classified as “high”.

Cell Proliferation Assays

For PHGDH or PSPH knockdown experiments, 10,000-20,000 MDA-MB-468, BT-20, HCC70, MCF-7, or MDA-MB-23 I cells were infected with shRNA-expressing lentiviruses of known titers at a multiplicity of infection of 2.5 to 5. Cells were cultured in 12-well plates and infected via a 30-minute spin at 2,250 RPM in a Beckman Coulter Allegra X-12R centrifuge with an SX4750 rotor and uPlate Carrier attachment followed by an overnight incubation in media containing polybrene. Eight days after infection the number of cells was determined using a Coulter Counter (Beckman) and used to calculate relative cell proliferation. Where indicated, standard RPMI media was supplemented with serine to concentrations 5-fold that of the serine already in the media. Where indicated, supplementation occurred at one and four days after lentiviral infection. For serine depletion experiments, cells were plated out as described above and the following day the standard culture medium was replaced with medium lacking serine or reconstituted with 1× serine. Dialyzed serum (3 kDa MWCO) was utilized in serine depletion experiments except in the case of MCF-10A cells, where standard 5% serum was utilized.

Immunohistochemistry and Immunoblotting

Immunoblotting was performed as described (11). PHGDH protein levels were quantified using an Odyssey Infrared Imager (Li-Cor). For each measurement, the PHGDH signal obtained was normalized to the RPS6 signal from the same lane after accounting for background fluorescence. Immunohistochemistry was performed on formalin fixed paraffin embedded sections using a boiling Dako antigen retrieval method, as described (12). Plastic Coplin jars containing a modified citrate buffer at pH 6.1 (Dako, Catalog S1699) were pre-heated in boiling water for ˜5 minutes. Slides were placed in the jars and heated in boiling water for 20 minutes, then removed from boiling water and cooled for at least 30 minutes at room temperature, then washed twice in distilled water. A 1:250 dilution of the PHGDH antibody was used. A pathologist scored, in a blinded fashion, the intensity of the PHGDH staining in the breast tumor samples using a scale of 0-3 that represents none/weak, moderate, and strong staining. About 70% of the samples exhibit strong staining. Use of the tumor samples for PHGDH staining was approved by Institutional Review Boards at the Massachusetts Institute of Technology (Protocol Number 1005003872) and Massachusetts General Hospital (Protocol Number 2010-P-001505/1).

### REFERENCES FOR METHODS

¹Moffat, J. et al., A lentiviral RNAi library for human and mouse genes applied to an arrayed viral high-content screen. Cell 124 (6), 1283-1298 (2006).
²Debnath, J. & Brugge, J. S., Modelling glandular epithelial cancers in three-dimensional cultures. Nat Rev Cancer 5 (9), 675-688 (2005).
³Rhodes, D. R. et al., Oncomine 3.0: genes, pathways, and networks in a collection of 18,000 cancer gene expression profiles. Neoplasia 9 (2), 166-180 (2007).
⁴Mikkelsen, T. S. et al., Dissecting direct reprogramming through integrative genomic analysis. Nature 454 (7200), 49-55 (2008).
⁵Kim, J., Chu, J., Shen, X., Wang, J., & Orkin, S. H., An extended transcriptional network for pluripotency of embryonic stem cells. Cell 132 (6), 1049-1061 (2008).
⁶Marson, A. et al., Connecting microRNA genes to the core transcriptional regulatory circuitry of embryonic stem cells. Cell 134 (3), 521-533 (2008).
⁷Ben-Porath, I, et al., An embryonic stem cell-like gene expression signature in poorly differentiated aggressive human tumors. Nat Genet. 40 (5), 499-507 (2008).
⁸Luo, B. et al., Highly parallel identification of essential genes in cancer cells. Proc Natl Acad Sci USA 105 (51), 20380-20385 (2008).
⁹Beroukhim, R. et al., The landscape of somatic copy-number alteration across human cancers. Nature 463 (7283), 899-905.
¹⁰van de Vijver, M. J. et al., A gene-expression signature as a predictor of survival in breast cancer. N Engl J Med 347 (25), 1999-2009 (2002).
¹¹Sancak, Y. et al., Ragulator-Rag complex targets mTORC1 to the lysosomal surface and is necessary for its activation by amino acids. Cell 141 (2), 290-303.
¹²Kalaany, N.Y. & Sabatini, D. M., Tumors with PI3K activation are resistant to dietary restriction. Nature 458 (7239), 725-731 (2009).

Those skilled in the art will recognize, or be able to ascertain using no more than routine experimentation, many equivalents to the specific embodiments of the invention described herein. The scope of the present invention is not intended to be limited to the embodiments described above. The invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Articles such as “a” and “an”, and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. The invention provides embodiments in which exactly one member of the group is present, employed in, or otherwise relevant to a given product or process. The invention also provides embodiments in which more than one, or all of the group members are present, employed in, or otherwise relevant to a given product or process. It is to be understood that the invention encompasses embodiments in which one or more limitations, elements, clauses, descriptive terms, etc., of a claim is introduced into another claim. For example, a claim that is dependent on another claim can be modified to include one or more elements or limitations found in any other claim that is dependent on the same base claim.

Where the claims recite a composition, it is understood that methods of using the composition as disclosed herein are provided, and methods of making the composition according to any of the methods of making disclosed herein are provided. Where the claims recite a method, it is understood that a composition for performing the method is provided. Where elements are presented as lists or groups, each subgroup is also disclosed. It should also be understood that, in general, where the invention, or aspects of the invention, is/are referred to as comprising particular elements, features, etc., certain embodiments of the invention or aspects of the invention consist of, or consist essentially of, such elements, features, etc.

It should also be understood that, unless clearly indicated to the contrary, in any methods claimed herein that include more than one step or act, the order of the steps or acts of the method is not necessarily limited to the order in which the steps or acts of the method are recited.

Where ranges are given herein, the invention provides embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment of the invention in which a numerical value is prefaced by “about”, the invention provides an embodiment in which the exact value is recited. In any embodiment of the invention in which a numerical value is not prefaced by “about”, the invention provides an embodiment in which the value is prefaced by “about”. Where the phrase “at least” precedes a series of numbers, it is to be understood that the phrase applies to each number in the list (it being understood that, depending on the context, 100% of a value may be an upper limit). It is also understood that any particular embodiment, feature, or aspect of the present invention may be explicitly excluded from any one or more of the claims or aspects or embodiments of the invention. For example, any gene, drug target, compound, compound class, compound combination, disease, tumor type, tumor characteristic, or therapeutic indication may be excluded.

SUPPLEMENTARY TABLE 2

| Gene Name | Gene Symbol | Entrez Gene ID | All Tumour | Breast | Stem Cell |
|---|---|---|---|---|---|
| ATP-binding cassette, sub-family A (ABC1), | ABCA1 | 19 | 1 | | |
| member 1 | | | | | |
| ATP-binding cassette, sub-family C | ABCC1 | 4363 | 1 | 1 | |
| (CFTR/MRP), member 1 | | | | | |
| ATP-binding cassette, sub-family C | ABCC4 | 10257 | 1 | 1 | 1 |
| (CFTR/MRP), member 4 | | | | | |
| ATP-binding cassette, sub-family C | ABCC5 | 10057 | 1 | | |
| (CFTR/IVIRP), member 5 | | | | | |
| ATP-binding cassette, sub-family E (OABP), | ABCE1 | 6059 | | 1 | 1 |
| member 1 | | | | | |
| ATP-binding cassette, sub-family G (WHITE), | ABCG1 | 9619 | 1 | | 1 |
| member 1 | | | | | |
| acyl-CoA, thioesterase 9 | ACOT9 | 23597 | | 1 | 1 |
| Aminoacylase 1 | ACY1 | 95 | 1 | | 1 |
| alanine-glyoxylate aminotransferase 2-like 2 | AGXT2L2 | 85007 | 1 | | |
| S-adenosylhomocysteine hydrolase | AHCY | 191 | 1 | | |
| adenylate kinase 2 | AK2 | 204 | 1 | 1 | |
| aldehyde dehydrogenase 18 family, member A1 | ALDH18A1 | 5832 | 1 | | |
| adenosylmethionine decarboxylase 1 | AMD1 | 262 | | 1 | 1 |
| aquaporin 9 | AQP9 | 366 | | 1 | |
| Asparagine synthetase | ASNS | 440 | | 1 | 1 |
| ATPase, class VI, type 11A | ATP11A | 23250 | | 1 | |
| ATPase, Ca++ transporting, cardiac muscle, | ATP2A2 | 488 | 1 | 1 | |
| slow twitch 2 | | | | | |
| ATPase, Ca++-sequestering | ATP2C1 | 27032 | 1 | 1 | |
| ATP synthase, H+ transporting, mitochondrial F0 | ATP5G3 | 518 | 1 | 1 | |
| complex, subunit c (subunit 9), isoform 3 | | | | | |
| Carbamoyl-phosphate synthetase 2, aspartate | CAD | 790 | 1 | 1 | |
| transcarbamylase, and dihydroorotase | | | | | |
| cystathionine-beta-synthase | CBS | 875 | | 1 | 1 |
| cytochrome c oxidase subunit Va | COX5A | 9377 | 1 | 1 | 1 |
| cytochrome c oxidase subunit VIb polypeptide 2 | COX6B2 | 125965 | | | 1 |
| (testis) | | | | | |
| cytochrome c oxidase subunit VIIa polypeptide 1 | COX7A1 | 1346 | | | 1 |
| (muscle) | | | | | |
| ceruloplasmin (ferroxidase) | CP | 1356 | 1 | 1 | |
| CTP synthase | CTPS | 1503 | | 1 | |
| cubilin (intrinsic factor-cobalamin receptor) | CUBN | 8029 | | | 1 |
| dihydrofolate reductase | DHFR | 1719 | | 1 | 1 |
| dehydrogenase E1 and transketolase domain | DHTKD1 | 55526 | | 1 | 1 |
| containing 1 | | | | | |
| deoxythymidylate kinase (thymidylate kinase) | DTYMK | 1841 | 1 | | 1 |
| enolase 1, (alpha) | ENO1 | 2023 | 1 | 1 | 1 |
| FAD1 flavin adenine dinucleotide synthetase | FLAD1 | 80308 | 1 | 1 | 1 |
| homolog (S. cerevisiae) | | | | | |
| folate receptor 1 (adult) | FOLR1 | 2348 | | 1 | 1 |
| glyceraldehyde-3-phosphate dehydrogenase | GAPDH | 2597 | 1 | 1 | 1 |
| phosphoribosylglycinamide formyltransferase, | GART | 2618 | 1 | 1 | 1 |
| phosphoribosylglycinamide synthetase, | | | | | |
| phosphoribosylaminoimidazole synthetase | | | | | |
| glutamine-fructose-6-phosphate transaminase 1 | GFPT1 | 2673 | 1 | | |
| glycine dehydrogenase (decarboxylating) | GLDC | 2731 | | | 1 |
| glutaminase | GLS | 2744 | 1 | 1 | |
| glutaminase 2 (liver, mitochondrial) | GLS2 | 27165 | | | 1 |
| GDP-mannose 4,6-dehydratase | GMDS | 2762 | 1 | 1 | |
| guanine monphosphate synthetase | GMPS | 8833 | 1 | 1 | |
| glucose phosphate isomerase | GPI | 2821 | 1 | 1 | 1 |
| glutathione S-transferase A4 | GSTA4 | 2941 | | | 1 |
| glutathione S-transferase pi | GSTP1 | 2950 | 1 | 1 | |
| hexokinase 3 (white cell) | HK3 | 3101 | | 1 | 1 |
| hydroxymethylbilane synthase | HMBS | 3145 | 1 | 1 | |
| heparan sulfate 2-O-sulfotransferase 1 | HS2ST1 | 9653 | 1 | 1 | |
| hydroxysteroid (17-beta) dehydrogenase 14 | HSD17B14 | 51171 | | | 1 |
| isocitrate dehydrogenase 2 (NADP+), | IDH2 | 3418 | 1 | 1 | |
| mitochondrial | | | | | |
| indoleamine 2,3-dioxygenase 1 | IDO1 | 3620 | | 1 | 1 |
| inositol(myo)-1(or 4)-monophosphatase 2 | IMPA2 | 3613 | | 1 | 1 |
| inositol 1,4,5-triphosphate receptor, type 3 | ITPR3 | 13710 | 1 | 1 | 1 |
| potassium channel, subfamily K, member 5 | KCNK5 | 8645 | | 1 | 1 |
| potassium channel tetramerisation domain | KCTD5 | 54442 | 1 | 1 | |
| containing 5 | | | | | |
| lactate dehydrogenase B | LDHB | 3945 | | 1 | 1 |
| lysophospholipase I | LYPLA1 | 10434 | 1 | | 1 |
| methylcrotonoyl-Coenzyme A carboxylase 2 | MCCC2 | 64087 | 1 | | |
| (beta) | | | | | |
| methylenetetrahydrofolate dehydrogenase | MTHFD2 | 10797 | 1 | 1 | 1 |
| (NADP+ dependent) 2, methenyltetrahydrofolate | | | | | |
| cyclohydrolase | | | | | |
| NADH dehydrogenase (ubiquinone) 1 alpha | NDUFA4L2 | 56901 | 1 | 1 | 1 |
| subcomplex, 4-like 2 | | | | | |
| non-metastatic cells 1, protein (NM23A) | NME1 | 4830 | 1 | 1 | |
| expressed in | | | | | |
| non-metastatic cells 2, protein (NM23A) | NME2 | 4831 | 1 | | |
| expressed in | | | | | |
| nucleoside phosphorylase | NP | 4860 | | 1 | 1 |
| N-acetylneuraminate pyruvate lyase | NPL | 80896 | 1 | 1 | 1 |
| (dihydrodipicolinate synthase) | | | | | |
| 5′-nucleotidase domain containing 2 | NT5DC2 | 64943 | 1 | 1 | |
| nudix (nucleoside diphosphate linked moiety X)- | NUDT1 | 4521 | 1 | 1 | 1 |
| type motif 1 | | | | | |
| nudix (nucleoside diphosphate linked moiety X)- | NUDT21 | 11051 | | 1 | 1 |
| type motif 21 | | | | | |
| nudix (nucleoside diphosphate linked moiety X)- | NUDT5 | 11164 | 1 | 1 | |
| type motif 5 | | | | | |
| 2′-5′-oligoadenylate synthetase 2, 69/71 kDa | OAS2 | 4939 | 1 | 1 | 1 |
| phosphoribosylaminoimidazole carboxylase, | PAICS | 10606 | 1 | 1 | 1 |
| phosphoribosylaminoimidazole | | | | | |
| succinocarboxamide synthetase | | | | | |
| phosphodiesterase 9A | PDE9A | 5152 | 1 | 1 | 1 |
| prenyl (decaprenyl) diphosphate synthase, | PDSS1 | 23590 | | 1 | |
| subunit 1 | | | | | |
| pyridoxal (pyridoxine, vitamin B6) kinase | PDXK | 8566 | 1 | 1 | |
| phosphofructokinase, platelet | PFKP | 5214 | 1 | 1 | 1 |
| phosphoglycerate kinase 1 | PGK1 | 5230 | 1 | 1 | |
| phosphoglycerate dehydrogenase | PHGDH | 26227 | | 1 | |
| pipecolic acid oxidase | PIPOX | 51268 | | | 1 |
| pyruvate kinase, muscle | PKM2 | 5315 | 1 | | 1 |
| phospholipase A2, group VII (platelet-activating | PLA2G7 | 7941 | 1 | 1 | |
| factor acetylhydrolase, plasma) | | | | | |
| paraoxonase 2 | PON2 | 5445 | 1 | | |
| peroxiredoxin 4 | PRDX4 | 10549 | 1 | 1 | |
| phosphoserine transaminase | PSAT | 29968 | | 1 | 1 |
| phosphoserine phosphatase | PSPH | 5723 | 1 | 1 | |
| pyrroline-5-carboxylate reductase 1 | PYCR1 | 5831 | 1 | | |
| ribonucleotide reductase M2 polypeptide | RRM2 | 6241 | 1 | 1 | 1 |
| selenophosphate synthetase 1 | SEPHS1 | 22929 | 1 | 1 | 1 |
| serine hydroxymethyltransferase 2 | SHMT2 | 6472 | 1 | 1 | |
| (mitochondrial) | | | | | |
| solute carrier family 11 (proton-coupled divalent | SLC11A1 | 6556 | | 1 | 1 |
| metal ion transporters), member 1 | | | | | |
| solute carrier family 12, member 7 | SLC12A7 | 10723 | 1 | | 1 |
| solute carrier family 12 (potassium/chloride | SLC12A8 | 84561 | 1 | 1 | |
| transporters), member 8 | | | | | |
| solute carrier family 15 (oligopeptide | SLC15A1 | 6564 | | 1 | 1 |
| transporter), member 1 | | | | | |
| solute carrier family 16 (monocarboxylic acid | SLC16A1 | 6566 | 1 | 1 | 1 |
| transporters), member 1 | | | | | |
| solute carrier family 16 (monocarboxylic acid | SLC16A3 | 9123 | 1 | 1 | |
| transporters), member 3 | | | | | |
| solute carrier family 25 (mitochondrial carrier, | SLC25A1 | 6576 | | 1 | 1 |
| citrate transporter), member 1 | | | | | |
| solute carrier family 25 (mitochondrial carrier, | SLC25A13 | 10165 | | 1 | 1 |
| adenine nucleotide translocator), member 13 | | | | | |
| solute carrier family 25, member 36 | SLC25A36 | 55186 | 1 | | 1 |
| solute carrier family 25, member 37 | SLC25A37 | 51312 | 1 | 1 | |
| solute carrier family 25 (mitochondrial carrier, | SLC25A5 | 292 | | 1 | |
| adenine nucleotide translocator), member 5 | | | | | |
| solute carrier family 26, member 9 | SLC26A9 | 115019 | | 1 | |
| solute carrier family 2 (facilitated glucose | SLC2A1 | 6513 | 1 | 1 | 1 |
| transporter), member 1 | | | | | |
| solute carrier family 2 (facilitated glucose | SLC2A3 | 6515 | | 1 | 1 |
| transporter), member 3 | | | | | |
| solute carrier family 2 (facilitated glucose | SLC2A5 | 6518 | 1 | 1 | |
| transporter), member 5 | | | | | |
| solute carrier family 34 (sodium phosphate), | SLC34A2 | 10568 | 1 | 1 | |
| member 2 | | | | | |
| solute carrier family 35 (UDP-galactose | SLC35A2 | 7355 | 1 | 1 | |
| transporter), member 2 | | | | | |
| solute carrier family 35, member F2 | SLC35F2 | 54733 | 1 | 1 | 1 |
| solute carrier family 38, member 1 | SLC38A1 | 81539 | 1 | | 1 |
| solute carrier family 43, member 3 | SLC43A3 | 29015 | 1 | 1 | |
| solute carrier family 44, member 1 | SLC44A1 | 23446 | 1 | | 1 |
| solute carrier family 44, member 2 | SLC44A2 | 57153 | | 1 | |
| solute carrier family 4 (anion exchanger), | SLC4A4 | 8671 | 1 | | |
| member 4 | | | | | |
| solute carrier family 5 (sodium-dependent | SLC5A6 | 8884 | | 1 | 1 |
| vitamin transporter), member 6 | | | | | |
| solute carrier family 6 (neurotransmitter | SLC6A8 | 6535 | | 1 | 1 |
| transporter, creatine), member 8 | | | | | |
| olute carrier family 7 (cationic amino acid | SLC7A1 | 6541 | 1 | 1 | |
| transporter, y+ system), member 1 | | | | | |
| solute carrier family 7 (cationic amino acid | SLC7A5 | 8140 | 1 | 1 | |
| transporter, y+ system), member 5 | | | | | |
| solute carrier organic anion transporter family, | SLCO4A1 | 28231 | | 1 | |
| member 4a1 | | | | | |
| spermine synthase | SMS | 6611 | 1 | | 1 |
| Superoxide dismutase 2, mitochondrial | SOD2 | 6648 | | 1 | 1 |
| Squalene epoxidase | SQLE | 6713 | 1 | 1 | |
| steroid-5-alpha-reductase, alpha polypeptide 1 | SRD5A1 | 6715 | 1 | 1 | |
| (3-oxo-5 alpha-steroid delta 4-dehydrogenase | | | | | |
| alpha 1) | | | | | |
| sulfatase 1 | SULF1 | 23213 | 1 | | 1 |
| sulfotransferase family, cytosolic, 1C, member 4 | SULT1C4 | 27233 | 1 | 1 | |
| transaldolase 1 | TALDO1 | 6888 | | 1 | 1 |
| transporter 1, ATP-binding cassette, sub-family | TAP1 | 6890 | 1 | 1 | |
| B (MDR/TAP) | | | | | |
| thymidine kinase 1, soluble | TK1 | 7083 | 1 | 1 | |
| triosephosphate isomerase 1 | TPI1 | 7167 | 1 | 1 | |
| transient receptor potential cation channel, | TRPM2 | 7226 | 1 | 1 | |
| subfamily M, member 2 | | | | | |
| tissue specific transplantation antigen P35B | TSTA3 | 7264 | 1 | | |
| Tweety homolog 3 (Drosophila) | TTYH3 | 80727 | 1 | 1 | |
| thymidylate synthetase | TYMS | 7298 | 1 | 1 | |
| uridine-cytidine kinase 2 | UCK2 | 7371 | 1 | 1 | 1 |
| UDP-glucose ceramide glucosyltransferase-like 1 | UGCGL1 | 56886 | 1 | 1 | |
| uridine plhosphorylase 1 | UPP1 | 7378 | | 1 | 1 |
| ubiquinol-cytochrome c reductase hinge protein | UQCRH | 7388 | | 1 | 1 |
| voltage-dependent anion channel 1 | VDAC1 | 7416 | 1 | | 1 |

1-14. (canceled)

15. A method of assessing the likelihood that a tumor is sensitive to serine biosynthesis pathway inhibition, the method comprising: determining the level of expression or copy number of the PHGDH gene in a sample obtained from the tumor, wherein an elevated level of expression or copy number indicates a significant likelihood that the tumor is sensitive to inhibition of the serine biosynthesis pathway.

16. The method of claim 15, wherein the tumor is a breast tumor or melanoma.

17. The method of claim 15, wherein the tumor is an ER negative breast tumor.

18. The method of claim 15, wherein determining the level of PHGDH expression comprises determining the level of PHGDH protein.

19. The method of claim 15, wherein determining the level of PHGDH expression comprises performing immunohistochemistry (IHC) on the sample to detect PHGDH protein.

20. The method of claim 15, wherein determining the level of PHGDH expression comprises performing immunohistochemistry on the sample to detect PHGDH protein, and wherein strong staining for PHGDH indicates that a significant likelihood that the tumor is sensitive to inhibition of the serine biosynthesis pathway.

21. The method of claim 15, wherein determining the level of PHGDH expression comprises determining the level of mRNA encoding PHGDH protein.

22-53. (canceled)

54. A method of identifying a potential drug target for anticancer therapy, the method comprising: (a) providing a pool of cells comprising multiple distinct populations of tumorigenic cells, wherein each of at least 5 distinct populations harbors in its genomic DNA an expression cassette encoding an RNAi agent that has sequence correspondence to a different target gene; (b) introducing the pool of cells into an animal host; (c) maintaining the animal host for a sufficient time period for a tumor to develop under conditions in which the RNAi agents are expressed during at least part of the time period; (d) harvesting at least a portion of the tumor; and (e) identifying an RNAi agent that became significantly depleted during tumor formation as compared with its abundance in the pool of cells of step (b), wherein the gene to which such RNAi agent has sequence correspondence is identified as a potential drug target for anticancer therapy.

55-80. (canceled)

81. A collection of retroviral RNAi vectors, wherein at least 50%, 60%, 70%, 80%, 90%, or more of the vectors comprise sequences encoding RNAi agents targeted to genes listed in Supplementary Table S2.

82. The collection of RNAi vectors of claim 81, wherein the vectors are retroviral vectors.

83. The collection of RNAi vectors of claim 81, wherein the vectors are lentiviral vectors.

84. A collection of vertebrate cells comprising the retroviral RNAi vectors of claim 81.

85-88. (canceled)
